(19) United States
(12) Patent Application Publication
(10) Pub. No.: US 2009/0221522 A1
(43) Pub. Date: Sep. 3, 2009

(54) METHODS TO CORRECT GENE SET EXPRESSION PROFILES TO DRUG SENSITIVITY

(75) Inventors: Manuel Hidalgo, Baltimore, MD (US); Antonio Jimeno, Englewood, CO (US); Aik Choon Tan, Highlands Ranch, CO (US)

Correspondence Address: BANNER & WITCOFF, LTD., 1100 13th STREET, N.W., SUITE 1200, WASHINGTON, DC, 20005-4051, US

(73) Assignee: The Johns Hopkins University, Baltimore, MD (US)

(21) Appl. No.: 12/372,373

(22) Filed: Feb. 17, 2009

Related U.S. Application Data
(63) Continuation of application No. PCT/US09/34056, filed on Feb. 13, 2009.
(60) Provisional application No. 61/065,667, filed on Feb. 14, 2008.
(60) Provisional application No. 61/035,503, filed on Mar. 11, 2008.
(60) Provisional application No. 61/118,740, filed on Dec. 1, 2008.

(51) Int. Cl. A61K 31/7068; C12Q 1/68; C40B 30/00; A61K 31/337; A61K 31/4353; A61P 35/00

(52) U.S. Cl. 514/49; 435/6; 506/7; 514/449; 514/291

(57) ABSTRACT

The present invention comprises a treatment approach based on gene set-expression signatures that systematically connects a sample to a profile from a reference database to extrapolate the most effective therapeutic agent. Further disclosed are methods to optimize combination treatments.

### CROSS-REFERENCE

This application claims the benefit of U.S. Provisional Application 61/065,667, filed Feb. 14, 2008 and titled “Gene Expression-based Perturbability Assay to Identify Novel Targets and to Personalize Anticancer Therapy”; U.S. Provisional Application 61/035,503, filed Mar. 11, 2008 and titled “Method of Using Molecular Mimicry to Connect Pathway-based Gene Expression Profiles to Drug Sensitivity”; and U.S. Provisional Application 61/118,740, filed Dec. 1, 2008 and titled “Method of Using Molecular Mimicry to Connect Pathway-based Gene Expression Profiles to Drug Sensitivity”; which applications are incorporated herein by reference. This application claims the benefit of PCT Patent Application PCT/US09/34056, filed Feb. 13, 2009 and titled “Methods to Connect Gene Set Expression Profiles to Drug Sensitivity,” which application is incorporated herein by reference.

### STATEMENT AS TO FEDERALLY SPONSORED RESEARCH

This invention was made with the support of the United States government under grant numbers CA129963 and CA116554 awarded by the National Institutes of Health. The U.S. government has certain rights in the invention.

### BACKGROUND OF THE INVENTION

Currently 98% or more of cancer patients receive therapy following a “one-size-fits-all” approach that is anatomically-driven. As each individual patient poses a different genetic aberrant background in his/her cancer, the one-size-fits-all treatment is usually a poor fit. For example, a pancreatic cancer patient will unequivocally be given gemcitabine even though the response rate for this agent is less than 20%. See Herrmann R, Bodoky G, Ruhstaller T, et al. Gemcitabine Plus Capecitabine Compared With Gemcitabine Alone in Advanced Pancreatic Cancer: A Randomized, Multicenter, Phase III Trial of the Swiss Group for Clinical Cancer Research and the Central European Cooperative Oncology Group. Journal of Clinical Oncology 25:2212-17 (2007); Storniolo A M et al. An investigational new drug treatment program for patients with gemcitabine. Cancer 85:1261-68 (1999). Indeed, cancer is the only top-five disease where no meaningful improvements in mortality have been observed in the last 30 years. Moreover, the current approach is very expensive. For example, the standard of care therapies for colorectal, lung and breast cancer cost between $50,000 and $120,000 per patient and year of treatment. Because many patients only have time for one or two lines of therapy, the current non-selective strategy deprives the patients of sufficient opportunities to explore other therapies.

Personalized medicine aims to identify the optimal balance between efficacy and tolerability for an individual patient. By recognizing the differences in the genetic makeup between individuals and the increased understanding to the biology of cancer, some progress has been made in identifying treatments based on the presence of biomarkers. See, e.g., Hanahan D and Weinberg R A. The Hallmarks of Cancer. Cell 100:57-70, 2000; Vogelstein B and Kinzler K W: Cancer genes and the pathways they control. Nat Med 10:789-799, 2004. A prime example of biomarker-driven treatment includes Her2 over-expression as a biomarker for directing treatment of trastuzumab (Herceptin™). Her2 is over-expressed in 20-30% of breast cancers and is associated with lower responsiveness to standard treatments and poorer outcome. However, even in Her2-positive breast cancer patients, the objective response is only about 34%. See Vogel C L, Cobleigh M A, Tripathy D, et al. Efficacy and Safety of Trastuzumab as a Single Agent in First-Line Treatment of HER2-Overexpressing Metastatic Breast Cancer. Journal of Clinical Oncology 2002; 20(3):719-26. Thus, the majority of patients with the biomarker remain resistant to trastuzumab. Furthermore, the one-size-fits-all philosophy still applies to Her2-negative patients.

An alternative approach for personalized cancer treatment includes the use of direct-patient cellular or xenograft models, as described by Rubio-Viqueira B, Jimeno A, Cusatis G, et al. An In vivo Platform for Translational Drug Development in Pancreatic Cancer. Clin Cancer Res 12:4652-61 (2006). The patient samples are treated with multiple anticancer agents in order to find the most effective drug for that patient. See Samson D J, Seidenfeld J, Ziegler K, Aronson N. Chemotherapy Sensitivity and Resistance Assays: A Systematic Review. Journal of Clinical Oncology 22:3618-30 2004; Schrag D, Garewal H S, Burstein H J, Samson D J, Von Hoff D D, Somerfield M R. American Society of Clinical Oncology Technology Assessment: Chemotherapy Sensitivity and Resistance Assays. Journal of Clinical Oncology 22:3631-38 2004. Accordingly, this approach aims to provide tailor-made treatments, where the optimal agent or agents are identified for a particular patient. This has been called “chemotherapy sensitivity and resistance assays.” However, these methods have not yet shown to be predictive of anticancer drug efficacy in the clinic. See Samson D J, Seidenfeld J, Ziegler K, Aronson N. Chemotherapy Sensitivity and Resistance Assays: A Systematic Review. Journal of Clinical Oncology 22:3618-30 2004; Schrag D, Garewal H S, Burstein H J, Samson D J, Von Hoff D D, Somerfield M R. American Society of Clinical Oncology Technology Assessment: Chemotherapy Sensitivity and Resistance Assays. Journal of Clinical Oncology 22:3631-38 2004. In addition, substantial pitfalls remain for these tailor-made approaches, as they are restricted to: 1) patients undergoing surgical resection of their cancers with availability of excess tumor tissue; and/or 2) successful propagation of the tumor cells in in vitro or in vivo conditions.

Although cancers are diverse, there are underlying similarities in the mechanisms used to acquire and maintain malignant properties, especially when looking at the higher hierarchical level of pathway organization. See Hanahan D, Weinberg R A. The Hallmarks of Cancer. Cell 100:57-70 (2000); Vogelstein B, Kinzler K W. Cancer genes and the pathways they control, Nat Med 10:789-99 (2004). Therefore, drugs developed specifically to target pathways in one cancer might have an impact on other anatomically unrelated cancers that show the same activated pathways. Identifying pathway signatures from a rich drug-screen panel and correlating these signatures with drug efficacy represents a powerful strategy to discover better ways to treat cancer. One recent approach of identifying pathway signatures includes engineering pathway deregulation cell lines in vitro, profiling gene expression in the cells and correlating the expression profiles to drug sensitivity and clinical outcomes. See, e.g., Bild A H, Yao G, Chang J T, et al. Oncogenic pathway signatures in human cancers as a guide to targeted therapies. Nature 439:353-57 (2006); Potti A, Dressman H K, Bild A, et al. Genomic signatures to guide the use of chemotherapeutics. Nat Med 12:1294-300 (2006). This approach seems promising but may not represent the true pathway signatures in tumors in vivo. See Watters J W, Roberts C J. Developing gene expression signatures of pathway deregulation in tumors. Mol Cancer Ther 5:2444-49 (2006).

Thus, there is a need for additional approaches for implementing personalized medicine.

### SUMMARY OF THE INVENTION

In one embodiment, the present invention provides a method for selecting a candidate therapeutic agent, comprising: (a) determining a gene set expression profile for two or more genes in a target cell; (b) comparing the gene set expression profile of the target cell to one or more gene set expression profiles of a panel of reference cells, wherein the panel comprises cells from more than two different cell types; (c) identifying a reference cell from the panel that has the most similar gene set expression profile to the target cell according to the comparison in step (b); and (d) selecting a therapeutic agent known for treating a condition in the reference cell identified in step c).

In another embodiment, the present invention provides a method for treating a subject in need thereof, comprising: (a) extracting a sample from the subject; (b) determining a gene set expression profile for two or more genes in a target cell derived from the sample in step (a); (c) comparing the gene set expression profile of the target cell to one or more gene set expression profiles of a panel of reference cells, wherein the panel comprises cells from more than two different cell types; (d) identifying a reference cell from the panel that has the most similar gene set expression profile to the target cell according to the comparison in step (c); and (e) treating the subject with one or more therapeutic agents known for treating a condition in the reference cell identified in step (d). In some embodiments, a first therapeutic agent is administered to the subject before step (a). In some embodiments, the first therapeutic agent comprises gemcitabine. In some embodiments, the one or more therapeutic agents used for treating the subject in step (e) are one or more of erlotinib, capecitabine, doxorubicine, docetaxel, etoposide, oxaliplatin, irinotecan or cisplatin.

In another embodiment, the present invention provides a method to select a subject for enrollment in a clinical trial of one or more therapeutic agents, comprising: (a) extracting a sample from a subject; (b) determining a gene set expression profile for two or more genes in a target cell derived from the sample in step (a); (c) comparing the gene set expression profile of the target cell to one or more gene set expression profiles of a panel of reference cells, wherein the panel comprises cells from more than two different cell types; (d) identifying a reference cell from the panel that has the most similar gene set expression profile to the target cell according to the comparison in step (c); and (e) selecting the subject for enrollment in the clinical trial if the one or more therapeutic agents are known for treating a condition in the reference cell identified in step (d).

In another embodiment, the present invention provides a method for predicting response of a subject to a particular therapeutic agent, comprising: (a) extracting a sample from the subject; (b) determining a gene set expression profile for two or more genes in a target cell derived from the sample in step (a); (c) comparing the gene set expression profile of the target cell to one or more gene set expression profiles of a panel of reference cells, wherein the panel comprises cells from more than two different cell types; (d) identifying a reference cell from the panel that has the most similar gene set expression profile to the target cell according to the comparison in step (c); and (e) predicting that the subject will respond to the therapeutic agent if the therapeutic agent is known for treating a condition in the reference cell identified in step d), or predicting that the subject will not respond to the therapeutic agent if the therapeutic agent is ineffective in treating a condition in the reference cell identified in step (d).

In some embodiments, determining the gene set expression profile of the target cell comprises amplifying nucleic acids extracted from the target cell by reacting the nucleic acids with a plurality of nucleotide probes. In some embodiments, the reaction products are hybridized to one or more DNA microarrays. In some embodiments, the amplification comprises a real-time polymerase chain reaction. In some embodiments, the gene set expression profile of the target cell is determined using protein expression levels.

In some embodiments, determining the gene set expression profile of the target cell comprises comparing the expression levels of pre-defined gene sets in the target cell against the expression levels of the same gene sets in the panel of reference cells. In some embodiments, determining the gene set expression profile of the target cell comprises Gene Set Enrichment Analysis (GSEA). In some embodiments, the gene sets comprise biological pathways. In some embodiments, the biological pathways are defined by the KEGG biological pathway definitions. In some embodiments, the comparison step comprises ranking the gene set expression profiles of the reference panel according to their similarity to the expression profile of the target cell. In some embodiments, the ranking uses Spearman's rank correlation analysis.

In some embodiments, the target cell is extracted from a mammalian subject. In some embodiments, the extraction is from a tumor biopsy. In some embodiments, the biopsy comprises a fine needle aspirate biopsy, a paraffin block, or a frozen sample.

In some embodiments, the target cell is a tumor cell. In some embodiments, the tumor is a pancreatic tumor or a breast tumor. In some embodiments, the panel of reference cells comprises tumor cells. In some embodiments, the panel comprises one or more cells from the NCI-60 cell lines.

In some embodiments, the most similar reference cell according to the identifying step is derived from a different anatomical origin as compared to the target cell.

In another embodiment, the present invention provides a method for selecting a candidate therapeutic agent comprising: (a) contacting a target cell with a first therapeutic agent; (b) determining a response of the target cell to the first therapeutic agent using expression profiling; and (c) selecting a second therapeutic agent based on the response of the target cell to the first therapeutic agent.

In another embodiment, the present invention provides a method for treating a subject in need thereof, comprising: (a) extracting a target cell from the subject; (b) contacting the target cell with a first therapeutic agent; (c) determining a response of the target cell to the first therapeutic agent using expression profiling; and (d) treating the subject with a second therapeutic agent based on the response of the target cell to the first therapeutic agent.

In some embodiments, the target cell has not previously been contacted with the first therapeutic agent. In some embodiments, the expression profiling comprises reacting nucleic acid extracted from the target cell with a plurality of nucleotide probes.

In some embodiments, determining the response of the target cell to the first therapeutic agent comprises: i) determining the expression level of multiple genes in the target cell after contacting the target cell with the first therapeutic agent; ii) determining the expression level of the same genes in an identical control cell that has not been contacted with the first therapeutic agent; iii) comparing the expression levels determined in step i) and step ii); and iv) identifying genes that are overexpressed or underexpressed in the target cell versus the control cell according to the comparison in step iii). In some embodiments, the second therapeutic agent is a known therapeutic for cells that overexpress or underexpress one of more of the genes identified in step iv). In some embodiments, the genes identified in step iv) are overexpressed by two-fold or more or underexpressed by one-half-fold or less. In some embodiments, the genes identified in step iv) are underexpressed or overexpressed at statistically significant levels in the target cell versus the control cell. In some embodiments, statistical significance is determined at a p-value of 0.05 or less. In some embodiments, statistical significance is determined at a p-value of 0.01 or less. In some embodiments, the p-values are corrected for multiple comparisons. In some embodiments, the set of multiple genes whose expression is determined comprises one or more genes that are known drug targets. In some embodiments, the expression levels of multiple genes in steps i) and ii) are normalized before the comparison in step iii) by subtracting from each the expression levels of housekeeping genes determined in the same experiment. In some embodiments, the housekeeping genes comprise UBC, HPRT and SDHA. In some embodiments, determining expression levels comprises amplifying nucleic acids extracted from the target cell by reacting the nucleic acids with a plurality of nucleotide probes. In some embodiments, the reaction products are hybridized to one or more DNA microarrays. In some embodiments, the amplification comprises a real-time polymerase chain reaction.

In other embodiments, determining the response of the target cell to the first therapeutic agent comprises: i) determining a gene set expression profile of the target cell after contacting the target cell with the first therapeutic agent; ii) determining a gene set expression profile of an identical control cell that has not been contacted with the first therapeutic agent; iii) comparing the gene set expression profiles determined in step i) and step ii); and iv) identifying gene sets that are differentially expressed in the target cell versus the control cell according to the comparison in step iii). In some embodiments, determining gene set expression profiles comprises determining concordant expression of pre-defined sets of genes. In some embodiments, the gene set expression profiles are determined using Gene Set Enrichment Analysis (GSEA). In some embodiments, the gene sets comprise biological pathways. In some embodiments, the biological pathways are defined according to the KEGG biological pathway definitions. In some embodiments, the second therapeutic agent selected in step (c) is known to treat cells having deregulated gene sets identified in step iv).

In some embodiments, the target cell is a tumor cell. In some embodiments, the tumor is a pancreatic tumor or a breast tumor. In some embodiments, the target cell is removed from a mammalian subject. In some embodiments, the target cell is removed from the subject using a fine needle aspirate biopsy. In some embodiments, the subject has not previously been treated with the first therapeutic agent. In some embodiments, the first therapeutic agent is gemcitabine. In some embodiments, the first and second therapeutic agents are administered sequentially or concurrently.

In some embodiments, one or more of the determining, comparing, identifying and selecting steps above is performed by a computer executable logic.

In another embodiment, the present invention provides a computer system for selecting a candidate therapeutic agent, wherein the computer system comprises computer executable logic for: (a) determining a gene set expression profile for two or more genes in a target cell; (b) comparing the gene set expression profile of the target cell to one or more gene set expression profiles of a panel of reference cells, wherein the panel comprises cells from more than two different cell types; (c) identifying a reference cell from the panel that has the most similar gene set expression profile to the target cell according to the comparison in step b); and (d) selecting a therapeutic agent known for treating a condition in the reference cell identified in step c). In some embodiments, the computer system accesses a reference database containing drug susceptibility data and gene expression data for a panel of reference cells. In some embodiments, the computer system accesses the database remotely.

In another embodiment, the present invention provides a computer system for selecting a candidate therapeutic agent, wherein the computer system comprises computer executable logic for: (a) determining a response of a target cell to a first therapeutic agent using one or more expression profiles; and (b) selecting a second therapeutic agent based on the response of the target cell to the first therapeutic agent. In another embodiment, the present invention provides a kit comprising: (a) one or more digital storage media comprising this computer executable logic; and (b) a plurality of nucleic acid probes to amplify mRNA of one or more genes that are known drug targets.

In another embodiment, the present invention provides a kit comprising one or more digital storage media comprising computer executable logic as described in any of the methods above.

The above disclosure generally describes the present invention. A more complete understanding is obtained by reference to the incorporated specific examples, which are provide for purposes of illustration only and are not intended to limit the scope of the invention.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates an example flow diagram for selecting a therapeutic agent according to the present invention.

FIG. 2 illustrates the Gene Set Connectivity Map (GS-CMAP) concept.

FIG. 2 depicts two different microarray formats.

FIG. 4 illustrates an example flow diagram for selecting combination therapies according to the present invention.

FIG. 5 illustrates a computer system according to the present invention.

FIG. 6 illustrates hierarchical clustering of thirty pancreatic tumors and the NCI-60 panel. The clustering was performed using gene-expression profiles (A) and pathway-expression profiles (B).

FIG. 7 illustrates connecting pancreatic cancer cell lines with the NCI-60 gemcitabine sensitivity through GS-CMAP. Left panel (A) indicates the MTT assays for twelve pancreatic cancer cell lines. Right panel (B) indicates the connection between the pancreatic cancer cell lines with the normalized mean −log₁₀(GI₅₀) graph for gemcitabine of the NCI-60 panel sorted by sensitivity. Seven of the eight sensitive pancreatic cancer cell lines were assimilated to gemcitabine sensitive NCI60 cell lines (indicated as green circles); and two out of four resistant pancreatic cancer cell lines were connected to the gemcitabine resistant NCI-60 cell line.

FIG. 8 illustrates GS-CMAP prediction of sensitive and resistant cases for docetaxel. A case assimilating with a sensitive cell line was sensitive (PANC265), and two cases similar to resistant cell lines were resistant (PANC215 and PANC185). The insert graphs illustrate the tumor growth curves for these cases. Red and blue lines represent control and treated xenografts, respectively. Error bars represent standard deviations. Similar data for paclitaxel is also shown.

FIG. 9 illustrates validation of drug prediction for rapamycin and temsirolimus in in vivo models. A targeted agent (temsirolimus) and a case (PANC219) assimilating with a sensitive cell line was sensitive, and a case (JH024) assimilating with a resistant cell line was resistant. The insert graphs illustrate the tumor growth curves for these cases. Red and blue lines represent control and treated xenografts, respectively. Error bars represent standard deviations.

FIG. 10 illustrates disease free survival (DFS) differences based on efficacy prediction according to the present invention. The median DFS for the predicted gemcitabine sensitive and resistant groups are 491 and 162 days, respectively (p=0.04).

FIG. 11 illustrates a pre-clinical design. Xenografts resistant to gemcitabine as first-line one-size-fits-all treatment defined as TGI >20% were randomly assigned as (i) control; (ii) erlotinib as a one-size-fits-all second-line treatment; and (iii) second-line treatment selected according to the present invention.

FIG. 12 illustrates validation on the xenografts of FIG. 11 based on prediction according to the present invention. Four xenograft cases were used. Three of the four xenografts responded to the selected choice of drug treatment, but only one xenograft responded to erlotinib, a one-size-fits-all treatment. A case is defined as a responder to the drug treatment if Tumor Growth Inhibition (TGI) is <20%. Negative TGI values indicate tumor regression.

FIG. 13 illustrates box plots for the three treatments plans from FIG. 12. The median TGI for gemcitabine, erlotinib and choice of drug according to the present invention (pret-a-porter) for these cases are 36.5%, 33% and 0%, respectively.

### DETAILED DESCRIPTION OF THE INVENTION

In one aspect, the present invention provides methods using molecular mimicry to connect gene expression data organized in the context of gene sets, e.g., biological pathways, with drug efficacy. In another aspect, the invention provides a gene expression-based perturbability assay to identify targets and personalize anticancer therapy.

Several key observations indicate that gene sets, e.g., biological pathways, provide a basis for developing such approaches. First, gene expression is measured accurately and has shown promise as the universal language in disease characterization and prognostication. Second gene expression is used to connect different biological states and systems. Finally, biological pathways drive disease phenotypes and, therefore, can be used as the connectable traits.

The present invention takes advantage of these premises by demonstrating that a given tumor is connected with another tumor based on gene set expression similarities and that drug response is similar in closely connected tumors. Accordingly, the present invention provides methods for connecting gene expression data organized in the context of gene sets with drug efficacy. In one aspect, the present invention discloses a method for personalized treatment by systematically connecting the most similar gene expression profile from a reference database of profiles and extrapolating the most effective drug for an individual subject. In another aspect, the present invention provides methods to select one or more second-line therapies using methods of the invention.

### 1. Gene Set Connection Approaches

In one aspect, the present invention provides methods for connecting gene expression data organized in the context of gene sets, e.g., biological pathways, with efficacy of one or more therapeutic agents. The method comprises determining a gene set expression profile, also referred to as a gene set-expression signature, for two or more genes in a target cell. The gene set expression profile of the target cell is compared to one or more gene set expression profiles for one or more reference cells, or a panel of reference cells, wherein the panel comprises cells from more than two different cell types.

In one embodiment, a reference cell is identified from a panel of reference cells that have the most similar gene set expression profile to the target cell. A therapeutic agent is selected that is known for treating a condition in the reference cell whose gene set expression profile is identified as most similar to that of the target cell.

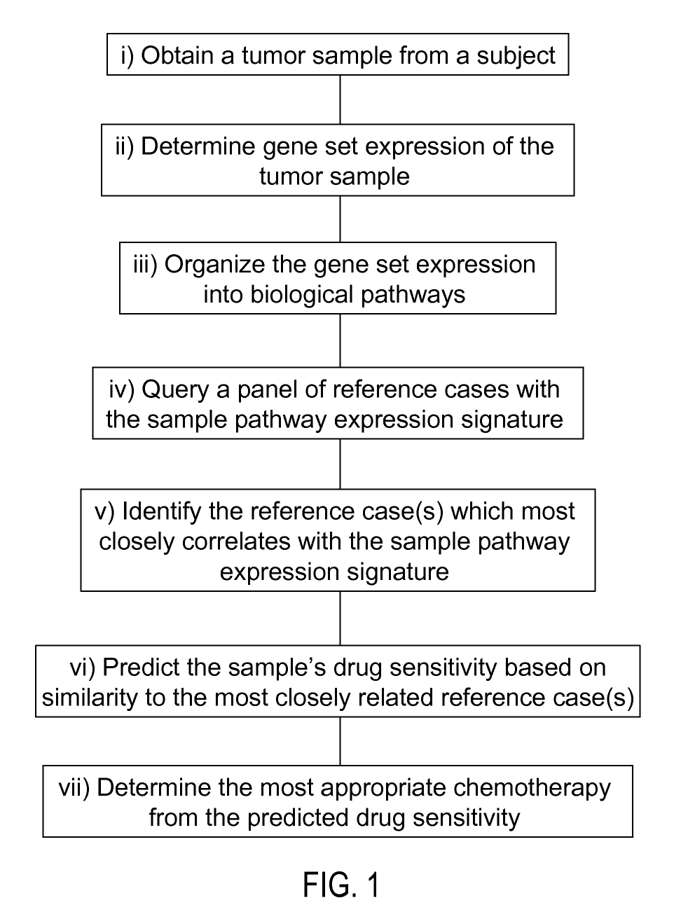

In one embodiment a subject is assessed for an appropriate chemotherapy regime (illustrated in FIG. 1). For example, assessing a subject comprises the steps of: (i) obtaining a tumor sample from a subject; (ii) determining a gene set expression of the tumor sample; (iii) organizing the gene set expression into biological pathways; (iv) querying a panel of reference cases with the sample pathway expression signature; (v) identifying the reference case(s) which most closely correlates with the sample pathway expression signature; (vi) predicting the sample's drug sensitivity based on similarity to the most closely related reference case(s); and (vii) determining the most appropriate chemotherapy from the predicted drug sensitivity. The methods of the present invention can be referred to as Gene Set Connectivity Mapping (GS-CMAP).

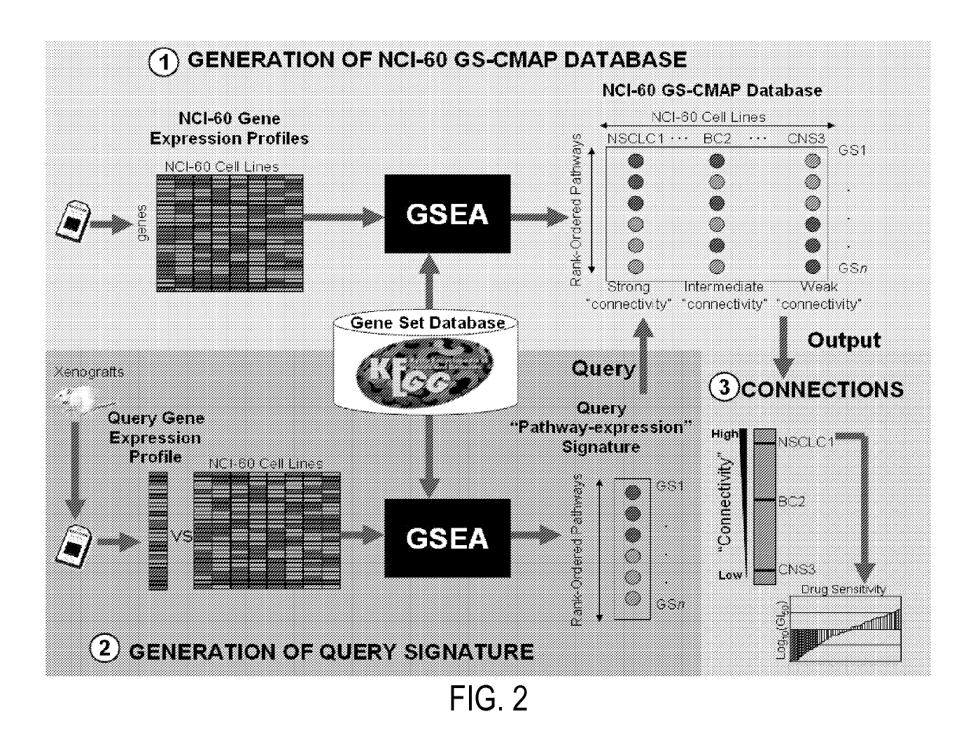

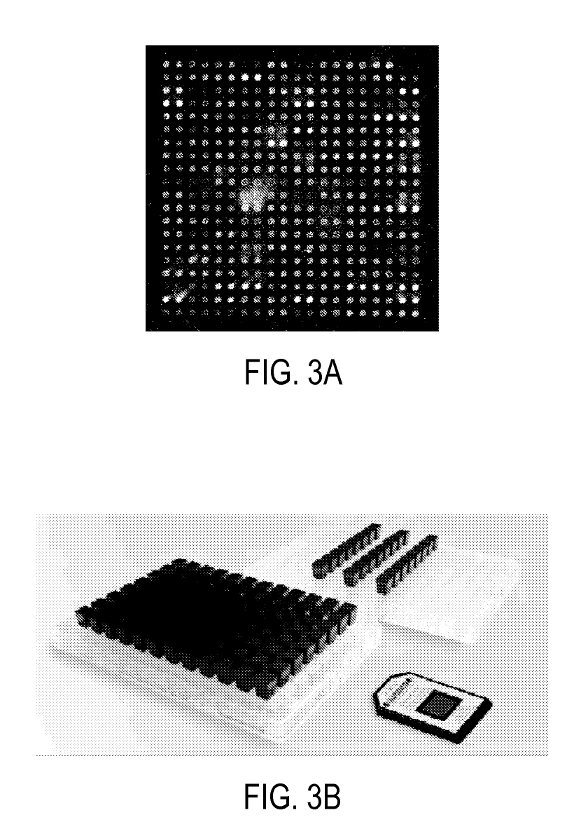

FIG. 2 illustrates another embodiment of the Gene Set Connectivity Map (GS-CMAP) concept. In this embodiment, the invention uses a reference database developed using the NCI-60 drug screening panel. The NCI-60 panel contains 60 diverse human cancer cell lines screened with more than 100,000 chemical compounds for anticancer activity since 1990 by the Developmental Therapeutics Program (DTP). See Huang R, Wallqvist A, Thanki N, Covell D G. Linking pathway gene expressions to the growth inhibition response from the National Cancer Institute's anticancer screen and drug mechanism of action. The Pharmacogenomics Journal 2005; 5:381-99; Staunton J E, Slonim D K, Coller H A, et al. Chemosensitivity prediction by transcriptional profiling. Proceedings of the National Academy of Sciences 2001; 98(19):10787-92; Covell D G, Huang R, Wallqvist A. Anticancer medicines in development: assessment of bioactivity profiles within the National Cancer Institute anticancer screening data. Mol Cancer Ther 6:2261-70 (2007); Potti A, Dressman H K, Bild A, et al. Genomic signatures to guide the use of chemotherapeutics. Nat Med 2006; 12(11): 1294-300.

As shown in FIG. 2, a method of the invention comprises assessing a tumor cell, e.g., a cell extracted from a subject or a cell derived from a cancer cell line and/or a xenograft, with a cell line from the NCI-60 panel using the reference database of drug sensitivity data. In some embodiments, connections are made using Gene Set Enrichment Analysis (GSEA) as described by Subramanian A, Tamayo P, Mootha V K, et al. Gene set enrichment analysis: A knowledge-based approach for interpreting genome-wide expression profiles. Proceedings of the National Academy of Sciences 2005; 102(43): 15545-50, and pathway correlation, using the Kyoto Encyclopedia of Genes and Genomes (KEGG), available online at www.genome.jp/kegg, as the base for systematic pathway classification. See Kanehisa M, Goto S, Hattori M, et al. From genomics to chemical genomics: new developments in KEGG. Nucl Acids Res 34(suppl_{—}1):D354-57 2006. Using this pathway-based approach, tumors are matched with a reference cell line and the tumor is assessed to show a similar pattern of susceptibility to anticancer agents as the reference cell line. Thus, one or more therapeutic agents are selected for treating the subject (i.e., target cell or target tumor) based on a therapeutic agent that is deemed effective against the reference cell line.

(a) Gene Expression Profiles

Gene expression profiling includes the measurement of the expression of multiple genes in a biological sample. For example, in one embodiment, the mRNA expression of thousands of genes may be determined at once. Alternately, in some embodiments, the mRNA expression of from between 1 to 500 genes is determined, or from between 1 to 10 genes, 2 to 20 genes, 5 to 25 genes, 10 to 50 genes, 20 to 100 genes, 50 to 200 genes, or 100 to 500 genes. Gene expression profiling measurements allow for a broad snapshot of the state of a biological sample. In some embodiments, expression levels from a cell are compared to another cell to identify genes that are differentially expressed. By way of example, the mRNA expression levels of a tumor cell are compared to those from a normal healthy but otherwise similar cell. Some genes are expressed at higher levels, i.e., upregulated or overexpressed, in the tumor cell compared to the expression of the same genes in the normal cell. Similarly, some genes are expressed at lower levels, i.e., downregulated or underexpressed, in the tumor cell compared to the expression of the same genes in the normal cell.

In some embodiments, identification of multiple genes that are upregulated in the tumor is used to create a signature to classify tumor types, e.g., according to origin or prognosis. In some embodiments, these approaches are extended to examine expression differences based upon any criteria, including different tissues, insult with drugs or other agents, response to various stimuli, etc. In various embodiments, one or more techniques are used to measure gene expression, including microarrays, polymerase chain reaction (PCR) techniques such as real-time PCR (RT-PCR), and serial analysis of gene expression (SAGE), subtractive hybridization and differential display. In some embodiments, expression profiling is via DNA micro array.

A microarray comprises a linear or two-dimensional or three dimensional (and solid phase) array of discrete regions, each having a defined area, formed on the surface of a solid support such as, but not limited to, glass, plastic, or synthetic membrane. The density of the discrete regions on a microarray is determined by the total numbers of immobilized polynucleotides to be detected on the surface of a single solid phase support. The arrays may contain less than about 500, less than about 1000, less than about 1500, less than about 2000, less than about 2500, less than about 3000, less than about 4000, less than about 5000, less than about 6000, less than about 7000, less than about 8000, less than about 10000, less than about 20000, less than about 30000, less than about 40000, less than about 50000, or less than about 60000 immobilized polynucleotides in total. In some embodiments, arrays can have more than about 60000 immobilized polynucleotides in total. A DNA microarray includes an array of oligonucleotide or polynucleotide probes placed on a chip or other surfaces used to hybridize to amplified or cloned polynucleotides from a sample. Since the position of each particular group of probes in the array is known, the identities of sample polynucleotides are determined based on their binding to a particular position in the microarray. As an alternative to the use of a microarray, an array of any size may be used in the practice of the invention, including an arrangement of one or more position of a two-dimensional or three dimensional arrangement in a solid phase to detect expression of a single gene sequence. In some embodiments, a microarray for use with the present invention may be prepared by photolithographic techniques (such as synthesis of nucleic acid probes on the surface from the 3′ end) or by nucleic synthesis followed by deposition on a solid surface.

The following U.S. patents teach methods of making and using oligonucleotide microarrays:
U.S. Pat. No. 5,837,832 Arrays of nucleic acid probes on biological chips
U.S. Pat. No. 5,770,722 Surface-bound, unimolecular, double-stranded DNA
U.S. Pat. No. 5,744,305 Arrays of materials attached to a substrate
U.S. Pat. No. 5,733,729 Computer-aided probability base calling for arrays of nucleic acid probes on chips
U.S. Pat. No. 5,631,734 Method and apparatus for detection of fluorescently labeled materials
U.S. Pat. No. 5,556,752 Surface-bound, unimolecular, double-stranded DNA

In some embodiments, gene expression is determined by hybridization of mRNA, or an amplified or cloned version thereof, of a sample cell to a polynucleotide that is unique to a particular gene sequence. Polynucleotides of this type contain about 16, about 18, about 20, about 22, about 24, about 26, about 28, about 30, or about 32 consecutive basepairs of a gene sequence that is not found in other gene sequences. Other embodiments include polynucleotides of at least or about 50, at least or about 100, about or 150, at least or about 200, at least or about 250, at least or about 300, at least or about 350, at least or about 400, at least or about 450, or at least or about 500 consecutive bases of a sequence that is not found in other gene sequences. The term “about” refers to an increase or decrease of 10% from the stated numerical value. Longer polynucleotides may contain minor mismatches (e.g., via the presence of mutations) which do not affect hybridization to the nucleic acids of a sample. Such polynucleotides may also be referred to as polynucleotide probes that are capable of hybridizing to sequences of the genes, or unique portions thereof, described herein. Such polynucleotides may be labeled to assist in their detection. The sequences may be those of mRNA encoded by the genes, the corresponding cDNA to such mRNAs, and/or amplified versions of such sequences. In some embodiments of the invention, the polynucleotide probes are immobilized on an array, other solid support devices, or in individual spots that localize the probes.

In various embodiments, commercially available microarrays are used in methods of the invention. FIG. 3 depicts two different microarray formats. FIG. 3A shows an example of a hybridized cDNA microarray. The circular spots correspond to hybridization probes or cDNAs arranged in a grid-like pattern. The brighter the spots, the more mRNA or other target has hybridized to the microarray, indicating higher levels of expression of the corresponding gene product. FIG. 3B shows an Affymetrix GeneChip® HT Human Genome U133 Array Plate Set (figure from corresponding product literature, available at www.affymetrix.com).

In other embodiments of the invention, all or part of a gene sequence may be amplified and detected by methods such as the polymerase chain reaction (PCR) and variations thereof, such as, but not limited to, quantitative PCR (Q-PCR), reverse transcription PCR (RT-PCR), and real-time PCR (including as a means of measuring the initial amounts of mRNA copies for each sequence in a sample), optionally real-time RT-PCR or real-time Q-PCR. Such methods would utilize one or two primers that are complementary to portions of a gene sequence, where the primers are used to prime nucleic acid synthesis. The newly synthesized nucleic acids are optionally labeled and may be detected directly or by hybridization to a polynucleotide of the invention. The newly synthesized nucleic acids may be contacted with polynucleotides (containing sequences) of the invention under conditions which allow for their hybridization. Additional methods to detect the expression of expressed nucleic acids include RNAse protection assays, including liquid phase hybridizations, and in situ hybridization of cells.

In other embodiments of the invention, gene expression is determined by analysis of expressed protein in a cell by use of one or more antibodies specific for one or more epitopes of individual gene products (proteins), or proteolytic fragments thereof, in the cell. The cell can be derived from various sources, as described herein, including but not limited to cell lines, bodily fluids, xenografts and biopsies. Detection methodologies suitable for use in the practice of the invention include, but are not limited to, immunohistochemistry of cell containing samples or tissue, enzyme linked immunosorbent assays (ELISAs) including antibody sandwich assays of cell containing tissues or blood samples, mass spectroscopy, and immuno-PCR. In some embodiments, analyzing protein content comprises assessing proteomic patterns, such as by mass spectrometry, chromatography, capillary electrophoresis, immunohistochemistry or 2-D gel electrophoresis. See, e.g., Latterich M, Abramovitz M, Leyland-Jones B. Proteomics: new technologies and clinical applications. Eur J. Cancer. 44:2737-41 (2008); Conrotto P, Souchelnytskyi S. Proteomic approaches in biological and medical sciences: principles and applications. Exp Oncol. 30:171-80 (2008). In other embodiments, reverse-phase protein lysate microarrays are used. See Paweletz, C. P., et al., Reverse phase protein microarrays which capture disease progression show activation of pro-survival pathways at the cancer invasion front. Oncogene 20:1981-1989 (2001).

(b) Gene Set Analysis

Gene set expression includes the expression of a plurality of genes, i.e., gene sets, which are coordinately up- or down regulated. Gene sets include groups of genes that share common biological function, chromosomal location, or regulation. Gene expression is assessed using standard molecular biology techniques as described herein. In some embodiments, gene expression is assessed with oligonucleotide microarrays. Gene sets comprise genes that are coordinately regulated, e.g., as part of a biological pathway.

(i) Gene Set Enrichment Analysis (GSEA)

In one embodiment, the present invention comprises Gene Set Enrichment Analysis (GSEA), a technique disclosed in Subramanian, Tamayo, et al. 2005, PNAS 102, 15554-15550; and Mootha, Lindgren, et al. 2003 Nat. Genet. 34, 267-273. In some embodiments, GSEA is performed by i) ranking genes in a data set, e.g., gene expression profiles of a DNA microarray analysis, based on their correlation to a chosen phenotype; ii) identifying all members of the gene set; and iii) calculating an Enrichment Score (ES), which can be a Normalized Enrichment Score (NES), representing the difference between the observed rankings and those that would be expected given a random distribution. After calculating the ES/NES, the method randomizes the sample labels and calculates the ES/NES for the gene set based on the random distribution. This process is repeated multiple times to create a distribution of randomized ES scores. Observed ES/NES scores that significantly outperform the randomized ES/NES scores are considered significant, thereby indicating that the given gene set is deregulated, i.e., up- or downregulated or differentially expressed, between cells having a certain biological phenotype. For example, the phenotype could be cancer and the gene set could the genes involved in the RAS pathway. The method can then be used to determine whether the RAS pathway is deregulated in the cancer cells compared to normal cells. Software to perform GSEA is freely available online at www.broad.mit.edu/gsea/msigdb/index.jsp.

Numerous alternative bioinformatics approaches have been developed to assess gene set expression profiles using gene expression profiling data. In various embodiments, any of these methods or similar methods are used in the present invention. Methods include but are not limited to those described in Segal, E. et al. Discovering molecular pathways from protein interaction and gene expression data. Nature Genet. 34:66-176 (2003); Segal, E. et al. A module map showing conditional activity of expression modules in cancer. Nature Genet. 36:1090-1098 (2004); Barry, W. T. et al. Significance analysis of functional categories in gene expression studies: a structure permutation approach. Bioinformatics 21:1943-1949 (2005); Tian, L. et al. Discovering statistically significant pathways in expression profiling studies. Proc Nat'l Acad Sci USA 102:13544-13549 (2005); Novak B A and Jain A N. Pathway recognition and augmentation by computational analysis of microarray expression data. Bioinformatics 22:233-41 (2006); Maglietta R et al. Statistical assessment of functional categories of genes deregulated in pathological conditions by using microarray data. Bioinformatics 23:2063-72 (2007); Bussemaker H J, Dissecting complex transcriptional responses using pathway-level scores based on prior information. BMC Bioinformatics 8 Suppl 6:S6 (2007).

(ii) Gene Sets

As described herein, GSEA and similar methods use gene sets to provide groups of genes that share common traits, e.g., biological function, chromosomal location, or regulation. The Molecular Signatures Database, http://www.broad.mit.edu/gsea/msigdb/index.jsp, lists over 5000 potential gene sets that can be used in GSEA or other techniques. These gene sets are segregated into five major categories (C1-C5) as follows:

TABLE 1
Molecular Signatures Database Gene Sets

| | Description |
|---|---|
| Gene Set | (see http://www.broad.mit.edu/gsea/msigdb/index.jsp) |
| C1: Positional Gene Sets | Gene sets corresponding to each human chromosome and each |
| | cytogenetic band that has at least one gene. (Cytogenetic locations were |
| | parsed from HUGO, October 2006, and Unigene, build 197. When there |
| | were conflicts, the Unigene entry was used.) These gene sets are helpful |
| | in identifying effects related to chromosomal deletions or amplifications, |
| | dosage compensation, epigenetic silencing, and other regional effects. |
| C2: Curated Gene Sets | Gene sets collected from various sources such as online pathway |
| | databases, publications in PubMed, and knowledge of domain experts. |
| | The gene set page for each gene set lists its source. |
| Canonical pathways | Gene sets from the pathway databases. Usually, these gene sets are |
| | canonical representations of a biological process compiled by domain |
| | experts. |
| Chemical and genetic | Gene sets that represent gene expression signatures of genetic and |
| perturbations | chemical perturbations. A number of these gene sets come in pairs: an |
| | xxx_UP (xxx_DN) gene set representing genes induced (repressed) by the |
| | perturbation. The gene set page for each gene set lists the PubMed |
| | citation on which it is based. |
| C3: Motif Gene Sets | Gene sets that contain genes that share a cis-regulatory motif that is |
| | conserved across the human, mouse, rat, and dog genomes. The motifs are |
| | catalogued in Xie, et al. (2005, Nature 434, 338-345) and represent |
| | known or likely regulatory elements in promoters and 3′-UTRs. These |
| | gene sets make it possible to link changes in a microarray experiment to a |
| | conserved, putative cis-regulatory element. |
| microRNA targets | Gene sets that contain genes that share a 3′-UTR microRNA binding |
| | motif. |
| Transcription factor targets | Gene sets that contain genes that share a transcription factor binding site |
| | defined in the TRANSFAC (version 7.4, http://www.gene- |
| | regulation.com/) database. Each of these gene sets is annotated by a |
| | TRANSFAC record. |
| C4: Computational Gene Sets | Computational gene sets defined by mining large collections of cancer- |
| | oriented microarray data. |
| Cancer gene neighborhoods | Gene sets defined by expression neighborhoods centered on 380 cancer- |
| | associated genes (Brentani, Caballero et al. 2003). This collection is |
| | identical to that previously reported in (Subramanian, Tamayo et al. |
| | 2005). |
| Cancer modules | Gene sets defined by Segal et al. (Nature Genetics 36, 1090 ? 1098, |
| | 2004). Briefly, the authors compiled gene sets (‘modules’) from a variety |
| | of resources such as KEGG, GO, and others. By mining a large |
| | compendium of cancer-related microarray data, they identified 456 such |
| | modules as significantly changed in a variety of cancer conditions. |
| C5: GO Gene Sets | Gene sets are named by GO term and contain genes annotated by that |
| | term. GSEA users: Gene set enrichment analysis identifies gene sets |
| | consisting of co-regulated genes; GO gene sets are based on ontologies |
| | and do not generally consist of co-regulated genes. |
| GO molecular function | Gene sets derived from the Molecular Function Ontology |
| | (http://www.geneontology.org/GO.function.guidelines.shtml). |
| GO biological process | Gene sets derived from the Biological Process Ontology |
| | (http://www.geneontology.org/GO.process.guidelines.shtml). |
| GO cellular component | Gene sets derived from the Cellular Component Ontology |
| | (http://www.geneontology.org/GO.component.guidelines.shtml). |

In some embodiments of the present invention, gene sets correspond to pathways including but not limited to one or more biological pathways, such as metabolic pathways, developmental pathways, signal-transduction pathways, genetic regulatory circuits or a combination thereof. In various embodiments, numerous sources of biological pathway gene sets are used, including but not limited to those disclosed in TABLE 2.

TABLE 2
Biological Pathway Databases

| Database | Description |
|---|---|
| GO Gene Sets | Gene sets are named by Gene Ontology (GO) term and contain genes |
| | annotated by that term. GO gene sets are based on ontologies and do not |
| | generally consist of co-regulated genes. |
| GO molecular function | Gene sets derived from the Molecular Function Ontology |
| | (http://www.geneontology.org/GO.function.guidelines.shtml). |
| GO biological process | Gene sets derived from the Biological Process Ontology |
| | (http://www.geneontology.org/GO.process.guidelines.shtml). |
| GO cellular component | Gene sets derived from the Cellular Component Ontology |
| | (http://www.geneontology.org/GO.component.guidelines.shtml). |
| UniPathway | UniPathway is a curated resource of metabolic pathways for the |
| | UniProtKB/Swiss-Prot knowledgebase. |
| | (http://www.grenoble.prabi.fr/obiwarehouse/unipathway) |
| BioCarta | (http://www.biocarta.com/genes/index.asp) |
| KEGG | Kyoto Encyclopedia of Genes and Genomes |
| | (http://www.genome.jp/kegg/) |
| MetaCyc | MetaCyc is a database of nonredundant, experimentally elucidated |
| | metabolic pathways. (http://metacyc.org/) |
| BioPAX: Biological Pathways | BioPAX is a collaborative effort to create a data exchange format for |
| Exchange | biological pathway data. (http://www.biopax.org/) |
| The Cancer Cell Map | The Cancer Cell Map contains selected cancer related signaling pathways |
| | which you can browse or search. (http://cancer.cellmap.org/cellmap/) |
| Reactome | A curated knowledgebase of biological pathways. (http://reactome.org/) |

One of skill in the art will appreciate that there are many other sources of gene sets and biological pathways that can be used in the present invention. Any gene set can be used to generate gene set expression signatures for use in the present invention.

In some embodiments, the pathways used in the present invention are those defined by the Kyoto Encyclopedia of Genes and Genomes (KEGG) database (Kanehisa M, Goto S, Hattori M, et al. From genomics to chemical genomics: new developments in KEGG. Nucl Acids Res 2006 34(suppl_{—}1):D354-7). The KEGG human pathways include metabolism, genetic information processing, environmental information processing, cellular processes and human diseases. Human pathway annotations downloaded from KEGG, or annotations for other gene set databases, are mapped to expression data, e.g., that derived from microarray experiments, in order to query the gene set expression in a given setting. For example, the KEGG gene annotations can be mapped to the Affymetrix HG-U133A, HG-U133B, and HG-U133 Plus 2.0 probe sets using the gene symbols available from the Affymetrix website (www.affymetrix.com). In other embodiments, one of skill in the art will appreciate that other biological pathway databases are used in the present invention.

(c) Reference Databases

In some embodiments, methods of the invention use a database of reference gene set signatures. In some embodiments, the database comprises biological data and therapeutic sensitivity data for a panel of biological samples. In some embodiments, the biological data is processed in such a way that it can be compared to similar data obtained from a target sample, e.g., a tumor sample. In some embodiments, the biological data is gene expression data. In some embodiments, the expression data is used to determine gene set expression profiles. In some embodiments, determining the gene set expression profile of the target cell comprises comparing the expression levels of pre-defined gene sets in the target cell against the expression levels of the same gene sets in the panel of reference cells. In one embodiment, Gene Set Enrichment Analysis (GSEA) is used to convert the gene expression data into gene set-expression profiles (signatures). In some embodiments, the biological data is processed by comparing “cell line i” versus “not cell line i,” to obtain a rank-ordered list of gene sets for cell line i, sorted by the normalized enrichment score (NES) of GSEA after a number of gene set permutations. In some embodiments, 500 gene set permutations are performed. In some embodiments, up to 100 gene set permutations are performed. In some embodiments, up to 200 gene set permutations are performed. In some embodiments, up to 300 gene set permutations are performed. In some embodiments, up to 400 gene set permutations are performed. In some embodiments, up to 500 gene set permutations are performed. In some embodiments, up to 600 gene set permutations are performed. In some embodiments, up to 700 gene set permutations are performed. In some embodiments, up to 800 gene set permutations are performed. In some embodiments, up to 900 gene set permutations are performed. In some embodiments, up to 1000 gene set permutations are performed. In some embodiments, more than 1000 gene set permutations are performed. In some embodiments, the Gene Sets are determined using biological pathway definitions, e.g., according to the KEGG database. The generated gene set expression pattern for each cell line, or reference signature i, can be stored in the reference database. The database can be updated when additional data is available.

The panel of biological samples comprises a panel of reference cases that may include cell lines, xenografts, direct-patient tumor samples, or other biological samples which have been assessed for gene set expression or other biological characteristics and sensitivity to at least one therapy. The cell types used for the panel comprise any number of different biological types. In some embodiments, different cell types comprise different cell lines. In some embodiments, different cell types comprise different cell lineages. In some embodiments, different cell types comprise different direct-patient tumor samples. In some embodiments, different cell types comprise cells from different anatomical origins, e.g., pancreas or breast, or any other. In some embodiments, different cell types comprise different samples. The samples can be derived from one source, e.g., one subject, or multiple sources, e.g., multiple subjects. In some embodiments, different cell types comprise cells having different diseased states. In some embodiments, different cell types comprise cells having different mutational status. In some embodiments, different cell types comprise cells having different genetic backgrounds. In some embodiments, different cell types comprise cells from different organisms. The cell types that makeup a panel can vary dramatically, e.g., comprising xenografts of breast cancer and cell lines of pancreatic cancer, or can be more closely related, e.g., a plurality of cell lines derived from differing breast tumor samples. Essentially, any panel of similar or different cells assessed with drug sensitivity data can be used in the methods of the present invention.

In some embodiments of the present invention, the NCI-60 cell lines are used to provide gene expression data and drug susceptibility data. Gene expression and drug sensitivity data are available for these cell lines as described herein.

(i) NCI-60 Cell Lines

The National Cancer Institute's NCI-60 cell lines comprise cells derived from nine different types of cancer (melanoma, leukemia, and cancers of the lung, colon, breast, prostate, kidney, ovary, and central nervous system). The cell lines included in the panel are listed in TABLE 3.

TABLE 3
NCI-60 cell lines.

| Cancer Type NCI-60 | Cell Line Name | NCI-60 ID |
|---|---|---|
| Breast | MCF7 | BC1 |
| Breast | NCI/ADR-RES | BC2 |
| Breast | MDA-MB-231/ATCC | BC3 |
| Breast | HS 578T | BC4 |
| Breast | MDA-MB-435 | BC5 |
| Breast | MDA-N | BC6 |
| Breast | BT-549 | BC7 |
| Breast | T-47D | BC8 |
| Colon | HT29 | CC1 |
| Colon | HCC-2998 | CC2 |
| Colon | HCT-116 | CC3 |
| Colon | SW-620 | CC4 |
| Colon | COLO 205 | CC5 |
| Colon | HCT-15 | CC6 |
| Colon | KM12 | CC7 |
| Central Nervous System | SNB-19 | CNS1 |
| Central Nervous System | SNB-75 | CNS2 |
| Central Nervous System | U251 | CNS3 |
| Central Nervous System | SF-268 | CNS4 |
| Central Nervous System | SF-295 | CNS5 |
| Central Nervous System | SF-539 | CNS6 |
| Leukemia | CCRF-CEM | LE1 |
| Leukemia | K-562 | LE2 |
| Leukemia | MOLT-4 | LE3 |
| Leukemia | HL-60(TB) | LE4 |
| Leukemia | RPMI-8226 | LE5 |
| Leukemia | SR | LE6 |
| Melanoma | LOX IMVI | ME1 |
| Melanoma | MALME-3M | ME2 |
| Melanoma | SK-MEL-2 | ME3 |
| Melanoma | SK-MEL-5 | ME4 |
| Melanoma | SK-MEL-28 | ME5 |
| Melanoma | M14 | ME6 |
| Melanoma | UACC-62 | ME7 |
| Melanoma | UACC-257 | ME8 |
| Non-Small Cell Lung | NCI-H23 | NSCLC1 |
| Non-Small Cell Lung | NCI-H522 | NSCLC2 |
| Non-Small Cell Lung | A549/ATCC | NSCLC3 |
| Non-Small Cell Lung | EKVX | NSCLC4 |
| Non-Small Cell Lung | NCI-H226 | NSCLC5 |
| Non-Small Cell Lung | NCI-H322M | NSCLC6 |
| Non-Small Cell Lung | NCI-H460 | NSCLC7 |
| Non-Small Cell Lung | HOP-62 | NSCLC8 |
| Non-Small Cell Lung | HOP-92 | NSCLC9 |
| Ovarian | OVCAR-3 | OC1 |
| Ovarian | OVCAR-4 | OC2 |
| Ovarian | OVCAR-5 | OC3 |
| Ovarian | OVCAR-8 | OC4 |
| Ovarian | IGROV1 | OC5 |
| Ovarian | SK-OV-3 | OC6 |
| Prostate | PC-3 | PC1 |
| Prostate | DU-145 | PC2 |
| Renal Cell | UO-31 | RC1 |
| Renal Cell | SN12C | RC2 |
| Renal Cell | A498 | RC3 |
| Renal Cell | CAKI-1 | RC4 |
| Renal Cell | RXF 393 | RC5 |
| Renal Cell | 786-0 | RC6 |
| Renal Cell | ACHN | RC7 |
| Renal Cell | TK-10 | RC8 |

(ii) NCI-60 Gene Expression Profiles

Publicly available NCI-60 gene expression profile data generated by Gene Logic, Inc. are available from the NCI Developmental Therapeutics Program (DTP) website (www.dtp.nci.nih.gov). The gene expressions of the NCI-60 cell lines were profiled by Affymetrix HG-U133A and HG-U133B GeneChip arrays which contain about 44,000 probes representing about 20,000 genes.

(iii) NCI-60 Drug Sensitivity Data

Drug sensitivity data for the NCI-60 cell lines is publicly available. This data contains 60 diverse human cancer cell lines screened with >100,000 chemical compounds for anticancer activity since 1990 by the Developmental Therapeutics Program (DTP). See Huang R, Wallqvist A, Thanki N, Covell D G. Linking pathway gene expressions to the growth inhibition response from the National Cancer Institute's anticancer screen and drug mechanism of action. The Pharmacogenomics Journal 2005; 5:381-99; Staunton J E, Slonim D K, Coller H A, et al. Chemosensitivity prediction by transcriptional profiling. Proceedings of the National Academy of Sciences 2001; 98(19):10787-92; Covell D G, Huang R, Wallqvist A. Anticancer medicines in development: assessment of bioactivity profiles within the National Cancer Institute anticancer screening data. Mol Cancer Ther 2007; 6(8):2261-70; Potti A, Dressman H K, Bild A, et al. Genomic signatures to guide the use of chemotherapeutics. Nat Med 2006; 12(11):1294-300.

In some embodiments, a reference database is created using the NCI-60 panel as follows. The NCI-60 drug sensitivity data, expressed in terms of the concentration of compound required for 50% growth inhibition (GI₅₀), was obtained from the NCI DTP website. For each drug/compound, a Z-score is computed for the log₁₀(GI₅₀) values across the NCI-60 cell lines by standardizing the 50% growth inhibition (GI₅₀) log values into 0 mean and 1 standard deviation (SD). In some embodiments, cell lines with Z-score at least 1 SD above the mean were defined as resistant to the compound, those with Z-score at least 1 SD below the mean were defined as sensitive, and cell lines with Z-score within 1 SD of the mean were considered to be intermediate. In other embodiments, cell lines with Z<−0.8, −0.8≦Z≦0.8, and Z>0.8 were defined as sensitive, intermediate and resistant, respectively. These methodologies are described in Lee J K, Havaleshko D M, Cho H, et al: A strategy for predicting the chemosensitivity of human cancers and its application to drug discovery. Proceedings of the National Academy of Sciences 104:13086-13091, 2007; Potti A, Dressman H K, Bild A, et al: Genomic signatures to guide the use of chemotherapeutics. Nat Med 12:1294-1300, 2006; Huang R, Wallqvist A, Thanki N, et al: Linking pathway gene expressions to the growth inhibition response from the National Cancer Institute's anticancer screen and drug mechanism of action. The Pharmacogenomics Journal 5:381-399, 2005; Covell D G, Huang R, Wallqvist A: Anticancer medicines in development: assessment of bioactivity profiles within the National Cancer Institute anticancer screening data. Mol Cancer Ther 6:2261-2270, 2007.

(d) Connection Methods

In some embodiments, a method is provided comprising connecting a pathway expression profile (signature) for a target, or query, cell to one or more reference pathway expression profiles (signatures). In some embodiments, connections are made by correlating pathway expression profiles. In some embodiments, correlation techniques include but are not limited to parametric and non-parametric methods or techniques based on mutual information and non-linear approaches. Some examples of parametric approaches include Pearson correlation (or Pearson r, also referred to as linear or product-moment correlation) and cosine correlation. Some examples of non-parametric methods include Spearman's Rank (or rank-order) correlation, Kendall's Tau correlation, and the Gamma statistic. Each correlation methodology can be used to determine the level of correlation between pathway expression signatures. For Pearson's correlation, the correlation coefficient r is used as the indicator of the level or degree of correlation. When other correlation methods are used, the correlation coefficient analogous to r may be used. In some embodiments, non-parametric techniques including Spearman's rank correlation are used to rank-order the reference signatures against the query signature. Kendall's Tau can also be used. Non-parametric methods are advantageous for some embodiments because they describe the relationship between two variables without making any assumptions about the frequency distribution of the variables. Positive and negative scores represent the positive and negative connectivity between the query and the reference samples.

In other embodiments, statistical classification techniques are used to group a target cell or the like with a similar reference cell. One of skill in the art will appreciate that a variety of methods are used to classify a target cell, e.g., using expression profiles. Such classification (pattern recognition) methods include, e.g., Bayesian classifiers, profile similarity, artificial neural networks, support vector machines (SVM), logistic or logic regression, linear or quadratic discriminant analysis, decision trees, clustering, principal component analysis, Fischer's discriminate analysis or nearest neighbor classifier analysis. Machine learning approaches to classification include, e.g., weighted voting, k-nearest neighbors, decision tree induction, support vector machines (SVM), and feed-forward neural networks. In some embodiments, Top Scoring Pairs (TSP) is used. See Tan, A C et al., Simple decision rules for classifying human cancers from gene expression profiles. Bioinformatics 21:3896-904 (2005). When basing classifiers on expression data for multiple genes, the classifiers are constructed using all genes or using only a subset of genes, e.g., only informative genes. In some embodiments, the subset of genes comprises those that show statistically different expression between groups of samples. In some embodiments, informative genes include those having sufficient expression levels in one or more expression profiles to be measured above background. Methods for determining statistical significance within the scope of the invention are described herein.

(e) Connectivity Mapping

In some embodiments, methods of the invention comprise determining a connectivity mapping to connect a target sample to a panel of one or more reference cells. FIG. 2 illustrates an embodiment for using Gene Set-Connectivity Mapping (GS-CMAP) to connect a query sample, e.g., a tumor sample, to the most similar reference sample. In this embodiment, NCI-60 reference signatures are generated in Step 1 using Gene Set Enrichment Analysis (GSEA), wherein the Gene Sets are determined using the KEGG biological pathway definitions. Using a gene set size filter criteria (min=10, max=500), 166 KEGG gene sets are analyzed. For each cell line i in the NCI-60 panel (see TABLE 3), GSEA is performed on two phenotypes, “cell line i” versus “not cell line i,” to obtain a rank-ordered list of pathways for cell line i, sorted by the normalized enrichment score (NES) of GSEA after 500 gene set permutations. This pathway pattern, known as the reference “pathway-signature i,” is unique for cell line i and is stored in a GS-CMAP database. In Step 2, the query gene expression profile, e.g., that of a target cancer cell or a xenograft, is compared to a set of reference gene expression profiles to generate the pathway-expression signature of the query sample using the same or similar permutation criteria as the reference set as described in Step 1. In some embodiments, this is done by comparing the gene expression profile of the query to those of the reference signatures, e.g., the NCI-60 panel as shown here. In other embodiments, the query's expression profile is compared to corresponding normal samples (e.g., healthy samples derived from the same anatomical origin as the query sample) to generate a pathway-expression signature. In Step 3, the pathway-expression signature of the query sample is connected to the GS-CMAP database. The rank-ordered list of pathways for the query is compared to each reference signature in the reference database to determine the similarity between the query and the reference samples. In this embodiment, the connection returns a rank-ordered list of NCI-60 cell lines wherein the top cell line is connected to the query. In this embodiment, Spearman's Rank correlation is used to compare the query and reference pathway expression signatures. Positive and negative scores represent the positive and negative connectivity between the query and the NCI-60 panel (Step 3). The top cell line from the list is determined as the connection between the query and NCI-60 panel. The query is linked to the drug sensitivity of the most similar cell line.

In some embodiments, the query is linked to the top 2 most similar cell lines. In some embodiments, the query is linked to the top 3 most similar cell lines. In some embodiments, the query is linked to the top 4 most similar cell lines. In some embodiments, the query is linked to the top 5 most similar cell lines. In some embodiments, the query is linked to the top 6 most similar cell lines. In some embodiments, the query is linked to the top 7 most similar cell lines. In some embodiments, the query is linked to the top 8 most similar cell lines. In some embodiments, the query is linked to the top 9 most similar cell lines. In some embodiments, the query is linked to the top 10 most similar cell lines. In some embodiments, the query is linked to more than 10 of the most similar cell lines. In some embodiments, the query is linked to all cell lines meeting a threshold criteria. In some embodiments, the threshold comprises a statistical significance value. In some embodiments, the significance value is a p-value less than or equal to 0.05. In some embodiments, the significance value is a p-value less than or equal to 0.01. In some embodiments, the significance value is a p-value less than or equal to 0.005. In some embodiments, the significance value is a p-value less than or equal to 0.001. In some embodiments, the significance value is a p-value less than or equal to 0.0005. In some embodiments, the significance value is a p-value less than or equal to 0.0001. In some embodiments, p-values are corrected for multiple comparisons. In some embodiments, multiple comparisons are corrected for using Bonferroni correction. In some embodiments, p-values are determined using permutation approaches, which are well known to those in the art. Permutation tests include randomization tests, re-randomization tests, exact tests, the jackknife, the bootstrap and other resampling schemes. In some embodiments, the threshold criterion comprises a correlation value. In some embodiments, the correlation value is r, as described herein. In some embodiments, r is greater than or equal to 0.95. In some embodiments, r is greater than or equal to 0.90. In some embodiments, r is greater than or equal to 0.85. In some embodiments, r is greater than or equal to 0.80. In some embodiments, r is greater than or equal to 0.75. In some embodiments, r is greater than or equal to 0.70. In some embodiments, r is greater than or equal to 0.65. In some embodiments, r is greater than or equal to 0.60. In some embodiments, r is greater than or equal to 0.55. In some embodiments, r is greater than or equal to 0.50. In some embodiments, r is greater than or equal to 0.45. In some embodiments, r is greater than or equal to 0.40. In some embodiments, r is greater than or equal to 0.35. In some embodiments, r is greater than or equal to 0.30. In some embodiments, r is greater than or equal to 0.25.

One of skill in the art will appreciate that a number of variations can be made without departing from the present invention. In some embodiments, reference panels of biological samples other than the NCI-60 are used to create a database of reference signatures to connect to the query signature. In some embodiments, gene sets other than the KEGG signatures are used to create the reference signatures from the reference panel. Many alternate gene sets are described herein, see TABLE 1, and any other gene set can be used. In some embodiments, the reference and query signatures are not derived from DNA microarray data, but the data derived from alternate techniques, e.g., RT-PCR or SAGE. In other embodiments, the gene sets are created from data derived from proteomics or other techniques. In some embodiments, statistical techniques other than GSEA are used to generate the signature for the reference and query samples, as disclosed herein. In some embodiments, statistical techniques other than Spearman's Rank are used to correlate the reference and query samples, as disclosed herein.

(f) Uses of Molecular Mimicry Approaches

(i) Individualized Anticancer Therapies

The present invention provides a method to select one or more therapeutic agents, e.g., for treating a target cell, e.g., a cancer cell. In one aspect, the present invention provides methods for connecting gene expression data organized in the context of gene sets, e.g., biological pathways, with efficacy of one or more therapeutic agents. The method comprises determining a gene set expression profile, also referred to as a gene set-expression signature, for two or more genes in a target cell. The gene set expression profile of the target cell is compared to one or more gene set expression profiles of a panel of reference cells, wherein the panel comprises cells from more than two different cell types. A reference cell is identified from the panel that has the most similar gene set expression profile to the target cell according to the comparison. A therapeutic agent is selected that is known for treating a condition in the reference cell whose gene set expression profile is identified as most similar to that of the target cell.

In another aspect, the present invention provides a method for treating a subject in need thereof. The method comprises extracting a sample from the subject. A gene set expression profile for two or more genes is determined for a target cell derived from the sample. The gene set expression profile of the target cell is compared to one or more gene set expression profiles of a panel of reference cells, wherein the panel comprises cells from more than two different cell types. A reference cell is identified from the panel that has the most similar gene set expression profile to the target cell according to the comparison. The subject is treated with a therapeutic agent known for treating a condition in the reference cell whose gene set expression profile is identified as most similar to that of the target cell.

In another aspect, the present invention provides a method to predict efficacy of a particular therapeutic agent. Thereby, the method can be used to determine whether to treat a subject with the therapeutic agent, e.g., a chemotherapeutic drug. The method comprises extracting a sample from the subject. A gene set expression profile, also referred to as a gene set-expression signature, for two or more genes is determined for a target cell derived from the sample. The gene set expression profile of the target cell is compared to one or more gene set expression profiles of a panel of reference cells, wherein the panel comprises cells from more than two different cell types. A reference cell is identified from the panel that has the most similar gene set expression profile to the target cell according to the comparison. The therapeutic agent is predicted to be efficacious if the therapeutic agent is known for treating a condition in the reference cell identified as most similar to the subject sample. Alternately, the therapeutic agent is predicted to be non-efficacious if the therapeutic agent is ineffective in treating a condition in the identified reference cell.

In some embodiments, determining the gene set expression profiles comprises reacting nucleic acids extracted from the target cell with a plurality of nucleotide probes. The reaction is used, e.g., to amplify mRNA from the target cell and thereby measure gene expression.

In some embodiments, expression levels are determined using a DNA microarray. In other embodiments, expression levels are determined using real-time PCR. In still other embodiments, SAGE is used. In still other embodiments, protein expression measurements are used to determine the gene set expression profile. In some embodiments, the gene set expression profile of the target cell is determined by comparing the expression levels of pre-defined gene sets in the target cell against the expression levels of the same gene sets in the panel of reference cells. In some embodiments, the gene set expression profile analysis comprises Gene Set Entichment Analysis (GSEA). Numerous alternate bioinformatics approaches can be used to analyze gene set expression profiles, as described herein. In some embodiments, gene sets correspond to biological pathways, such as metabolic pathways, developmental pathways, signal-transduction pathways, and genetic regulatory circuits. In some embodiments, gene sets are selected as defined by the KEGG biological pathway database. In some embodiments, gene sets are selected as defined by the GO ontologies. Gene sets can further include groups of genes that share common traits, e.g., biological function, chromosomal location, or regulation. Any gene set comprising groups of genes can be used. In some embodiments, the gene set expression profiles are compared by ranking the gene set expression profiles of the reference panel according to their similarity to the expression profile of the target cell. The comparison can be performed using a non-parametric statistical approach, such as Spearman's Rank. Alternate methods, such as other non-parametric approaches or parametric methods such as Pearson's correlation, can be used as well.

In some embodiments, the panel of reference cells comprises tumor cells. In some embodiments, the cells are derived from cell lines or xenografts. In some embodiments, the reference cells comprise the cells contained with the NCI-60 reference panel, as described herein.

The methods of the present invention can be performed on a variety of biological samples as disclosed herein. In some embodiments, the sample is a tumor cell. For example, the tumor cell can be a pancreatic tumor cell or a breast cancer tumor cell. The tumor cell can be derived from a cell line, a xenograft, directly from a patient, or from other sources. In some embodiments, the target cell is extracted from a mammalian subject. For example, the target cell can be extracted from a biopsy resected from a subject. The target cell can also be extracted using fine needle aspirate biopsy. In some embodiments, the samples are fresh. In other embodiments, the samples are frozen. In some embodiments, the samples are fixed, e.g., in paraffin blocks.

Based on the comparison step, as described herein, a reference cell from the panel is identified wherein the gene set expression profile of the identified reference cell is the most similar to the gene set expression profile of the target cell. The panel of reference cells can comprise cells from more than two different cell types. In some embodiments, the panel of reference cells comprises one or more cells selected from the NCI-60 cell lines. In some embodiments, the reference cell includes more than one cell that correlates with the target sample. For example, the method of the present invention can be used to select a therapeutic agent known to treat either of the top 2 reference cells whose expression profiles are most similar to the target cell. In some embodiments, agents are selected that treat one or more of the top 3 reference cells whose expression profiles are most similar to the target cell. In some embodiments, agents are selected that treat one or more of the top 4 reference cells whose expression profiles are most similar to the target cell. In some embodiments, agents are selected that treat one or more of the top 5 reference cells whose expression profiles are most similar to the target cell. In some embodiments, agents are selected that treat one or more of the top 6 reference cells whose expression profiles are most similar to the target cell. In some embodiments, agents are selected that treat one or more of the top 7 reference cells whose expression profiles are most similar to the target cell. In some embodiments, agents are selected that treat one or more of the top 8 reference cells whose expression profiles are most similar to the target cell. In some embodiments, agents are selected that treat one or more of the top 9 reference cells whose expression profiles are most similar to the target cell. In some embodiments, agents are selected that treat one or more of the top 10 reference cells whose expression profiles are most similar to the target cell. In some embodiments, agents are selected that treat one or more reference cell whose expression profiles correlates highly, e.g., shows a positive correlation or rank analysis, with the target cell.

(ii) Non-Anatomical Predictive Ability

In further embodiments, methods are provided comprising selecting one or more therapeutic agents for types of cancers or other diseases that are not included in the reference panel. The method comprises determining a gene set expression profile, also referred to as a gene set-expression signature, for two or more genes in a target cell. The gene set expression profile of the target cell is compared to one or more gene set expression profiles of a panel of reference cells, wherein the panel comprises cells from more than two different cell types. A reference cell is identified from the panel that has the most similar gene set expression profile to the target cell according to the comparison. A therapeutic agent is selected that is known for treating a condition in the reference cell whose gene set expression profile is identified as most similar to that of the target cell. The reference cell need not be derived from a similar origin, e.g. from the same anatomical origin, as the target cell.

For example, the NCI-60 cell lines as a reference set and a group of direct pancreatic cancer xenografts as individual, independent test cases, the method connected a colon cancer cell line to pancreatic cancer xenografts. See EXAMPLE 4. Pancreatic cancer is not included in the NCI-60 panel, but the present invention correctly predicted the response of pancreatic cancer cells to therapeutic agents. One of skill in the art will appreciate that connecting cells by the methods of the present invention is not limited to any particular biological origin. Samples comprising any of the cancer or tumor types disclosed herein, or others, can be connected to any of the cancer or tumor types disclosed herein, or others.

It will be understood by one of skill in the art that the same predictive ability can be used to select treatments for diseases that differ from the target cell and the panel of reference cells.

(iii) Selection of Structurally Similar Therapeutic Agents

The present invention provides a method to select one or more therapeutic agents that are structurally related to the selected agent, as disclosed herein. For example, the reference database may contain drug susceptibility to a first drug or other therapeutic agent, but a modified or otherwise improved version of that drug or agent is available. In such a case, the present invention affords a method to select the improved version of the drug.

The method comprises determining a gene set expression profile, also referred to as a gene set-expression signature, for two or more genes in a target cell. The gene set expression profile of the target cell is compared to one or more gene set expression profiles of a panel of reference cells, wherein the panel comprises cells from more than two different cell types. A reference cell is identified from the panel that has the most similar gene set expression profile to the target cell according to the comparison. A therapeutic agent is selected that is structurally or otherwise related to another agent known for treating a condition in the reference cell whose gene set expression profile is identified as most similar to that of the target cell. The selected therapeutic agent can be used in many aspects of the invention, e.g., to treat a subject in need thereof.

(iv) Selection of Standard and Non-Standard Therapeutic Agents

As described herein, the methods of the present invention can be used to select treatments that are known for use with the target cell. In some embodiments, the agents selected by the present invention represent the standard of care for a particular diseased condition. One of skill in the art will appreciate that the present invention can be used to select non-standard therapeutic agents to treat the target cell.

In a series of non-limiting example embodiments, three anti-cancer agents were used to treat xenografts from pancreatic origin using the methods of the present invention. First, the method was used to predict sensitivity of gemticabine, an approved cytotoxic drug for pancreatic cancer. Example 6 shows a statistically significant relationship between the drug sensitivity of connected reference cells and the connected pancreatic cancer xenografts. In another example, the method was used to predict sensitivity to docetaxel, an anti-microtubule agent approved for lung, head and neck, prostate and breast cancers. Example 5 shows that the present invention correctly predicted the response of pancreatic cancer xenografts to docetaxel. In the third example embodiment, the method was used to predict sensitivity of pancreatic xenografts to temsirolimus, a rapamycin pro-drug inhibitor of mTOR that has been recently approved for renal cell cancer treatment. Example 8 shows that the present invention correctly predicted response of the xenografts to this targeted agent. Thus, the method can identify optimal but non-standard therapeutic agents.

(v) Second-Line or Combination Treatments

The present invention provides a method to select one or more therapeutic agents to treat cancer after a subject has first been treated with one or more other therapeutic agents. The method comprises extracting a sample from a subject who has been treated previously with one or more agents. A gene set expression profile, also referred to as a gene set-expression signature, for two or more genes is determined for a target cell derived from the sample. The gene set expression profile of the target cell is compared to one or more gene set expression profiles of a panel of reference cells, wherein the panel comprises cells from more than two different cell types. A reference cell is identified from the panel that has the most similar gene set expression profile to the target cell according to the comparison. One or more therapeutic agents are selected that are known for treating a condition in the identified reference cell. The selected “second-line” therapeutic agent may not be the same as the one or more other therapeutic agents previously administered to the subject.

In some embodiments, the second-line therapeutic agent or agents are administered concurrently with that of the initial therapies in order to boost treatment response. In other embodiments, the second-line therapeutic agent or agents are administered sequentially, or after, to the initial therapies.

(vi) Clinical Trial Enrollment

In one embodiment, a method of the invention comprises selecting one or more subjects for enrollment into clinical trials of therapeutic agents. The method comprises extracting a sample from the subject. A gene set expression profile, also referred to as a gene set-expression signature, for two or more genes is determined for a target cell derived from the sample. The gene set expression profile of the target cell is compared to one or more gene set expression profiles of a panel of reference cells, wherein the panel comprises cells from more than two different cell types. A reference cell is identified from the panel that has the most similar gene set expression profile to the target cell according to the comparison. The subject is selected for enrollment in the clinical trial if the one or more therapeutic agents tested in the clinical trial are known for treating a condition in the reference cell identified as most similar to the subject sample.

Therefore, in some embodiments, methods of the invention comprise enriching early and proof-of-concept clinical trials by identifying subjects more likely to respond to the therapies at issue in the trials.

### 2. Perturbability Assay for Combination Therapy

In another aspect, the invention provides a gene expression-based perturbability assay to identify targets and personalize combination anticancer therapies. Perturbation of a biological system includes alteration of function induced by external or internal mechanisms. In one embodiment, the present invention discloses a method to select a candidate therapeutic agent, the method comprising contacting a target cell with a first therapeutic agent (thereby inducing a perturbation), determining a response of the target cell to the first therapeutic agent using expression profiling, and selecting a second therapeutic agent based on the response of the target cell to the first therapeutic agent. In some embodiments, the target cell the target cell has not previously been contacted with the first therapeutic agent.

In another aspect, the present invention discloses a method for treating a subject in need thereof, comprising extracting a target cell from the subject, contacting the target cell with a first therapeutic agent, determining a response of the cell to the first therapeutic agent using expression profiling, and treating the subject with a second therapeutic agent based on the response of the target cell to the first therapeutic agent. In some embodiments, the target cell has not previously been contacted with the first therapeutic agent. In some embodiments, the subject has not previously been treated with the first therapeutic agent before the method is performed. The assay allows identification of the most optimal drug to give to the subject in combination with, or after, the first therapeutic agent. In some embodiments, the first therapeutic agent corresponds to the standard of care for a given disease. In some embodiments, the first and second therapeutic agents are administered sequentially or concurrently. In some embodiments, the subject is treated sequentially with the second therapeutic agent after treating the subject with the first therapeutic agent. In other embodiments, the subject is treated concurrently with the first therapeutic agent and the second therapeutic agent. In some embodiments, the first therapeutic agent is gemcitabine. In that case, the second therapeutic agent can provide an optimal combination therapy for use with gemcitabine.

In some embodiments, the response of the target cell to the first therapeutic agent is determined by reacting nucleic acid extracted from the target cell with a plurality of nucleotide probes. The reaction is used, e.g., to amplify mRNA from the target cell and thereby measure gene expression. In some embodiments, expression levels are determined by a microarray experiment. In some embodiments, microarrays comprise low density microarrays. In other embodiments, expression levels are determined using techniques such as SAGE or RT-PCR. In still other embodiments, proteomic methods for measuring protein expression are used to determine the response of the target cell to the first therapeutic agent.

In some embodiments, the response of the target cell to the first therapeutic agent is assessed by determining the expression level of multiple genes in the target cell after contacting the target cell with the first therapeutic agent, determining the expression level of the same genes in an identical control cell removed from the subject that has not been contacted with the first therapeutic agent, comparing the expression levels determined in the previous steps, and identifying genes that are differentially expressed in the target cell versus the control cell. Differential expression comprises both overexpression (upregulation) and underexpression (downregulation). In some embodiments, the target cell can overexpress these genes compared to control cells at a level of 2-fold or higher. In some embodiments, the target cell can overexpress these genes compared to control cells at a level of 3-fold or higher. In some embodiments, the target cell can overexpress these genes compared to control cells at a level of 4-fold or higher. In some embodiments, the target cell can overexpress these genes compared to control cells at a level of 5-fold or higher. In some embodiments, the target cell can overexpress these genes compared to control cells at a level of 6-fold or higher. In some embodiments, the target cell can overexpress these genes compared to control cells at a level of 7-fold or higher. In some embodiments, the target cell can overexpress these genes compared to control cells at a level of 8-fold or higher. In some embodiments, the target cell can overexpress these genes compared to control cells at a level of 9-fold or higher. In some embodiments, the target cell can overexpress these genes compared to control cells at a level of 10-fold or higher.

In a likewise fashion, the target cell can downregulate, or underexpress, the genes compared to the control cells. In some embodiments, the target cell can underexpress these genes compared to control cells at a level of ½-fold or less. In some embodiments, the target cell can underexpress these genes compared to control cells at a level of ⅓-fold or less. In some embodiments, the target cell can underexpress these genes compared to control cells at a level of ¼-fold or less. In some embodiments, the target cell can underexpress these genes compared to control cells at a level of ⅕-fold or less. In some embodiments, the target cell can underexpress these genes compared to control cells at a level of ⅙-fold or less. In some embodiments, the target cell can underexpress these genes compared to control cells at a level of 1/7-fold or less. In some embodiments, the target cell can underexpress these genes compared to control cells at a level of ⅛-fold or less. In some embodiments, the target cell can underexpress these genes compared to control cells at a level of 1/9-fold or less. In some embodiments, the target cell can underexpress these genes compared to control cells at a level of 1/10-fold or less.

In other embodiments, the genes that are differentially expressed are determined using statistical techniques that are well known to those of skill in the art. One such technique includes Significance Analysis of Microarrays (SAM) and modifications thereof for determining whether changes in gene expression are statistically significant. See Tusher, V. G., R. Tibshirani, et al. Significance analysis of microarrays applied to the ionizing radiation response. Proceedings of the National Academy of Sciences 98:5116-5121 (2001); Dinu, I. P., et al., Improving gene set analysis of microarray data by SAM-GS. BMC Bioinformatics 8: 242 (2007). Other methods suitable for group comparison include nonparametric Wilcoxon signed-rank test, two-sample independent Student's t-test, F-test, Welch test, Chi-square, Kolmogorov-Smimov test, Mann-Whitney U-test (rank-sum test), ANOVA, parametric, semi-parametric or non-parametric regression modeling, or similar. Genes whose expression is identified as significantly different between the target and control samples (e.g., according to a statistical significance value) can be used to identify therapeutic agents. In some embodiments, the significance value is a p-value less than or equal to 0.05. In some embodiments, the significance value is a p-value less than or equal to 0.01. In some embodiments, the significance value is a p-value less than or equal to 0.005. In some embodiments, the significance value is a p-value less than or equal to 0.001. In some embodiments, the significance value is a p-value less than or equal to 0.0005. In some embodiments, the significance value is a p-value less than or equal to 0.0001. In some embodiments, p-values are corrected for multiple comparisons. In some embodiments, multiple comparisons are corrected for using Bonferroni correction. In some embodiments, p-values are determined using permutation approaches, which are well known to those in the art. Permutation tests include randomization tests, re-randomization tests, exact tests, the jackknife, the bootstrap and other resampling schemes.

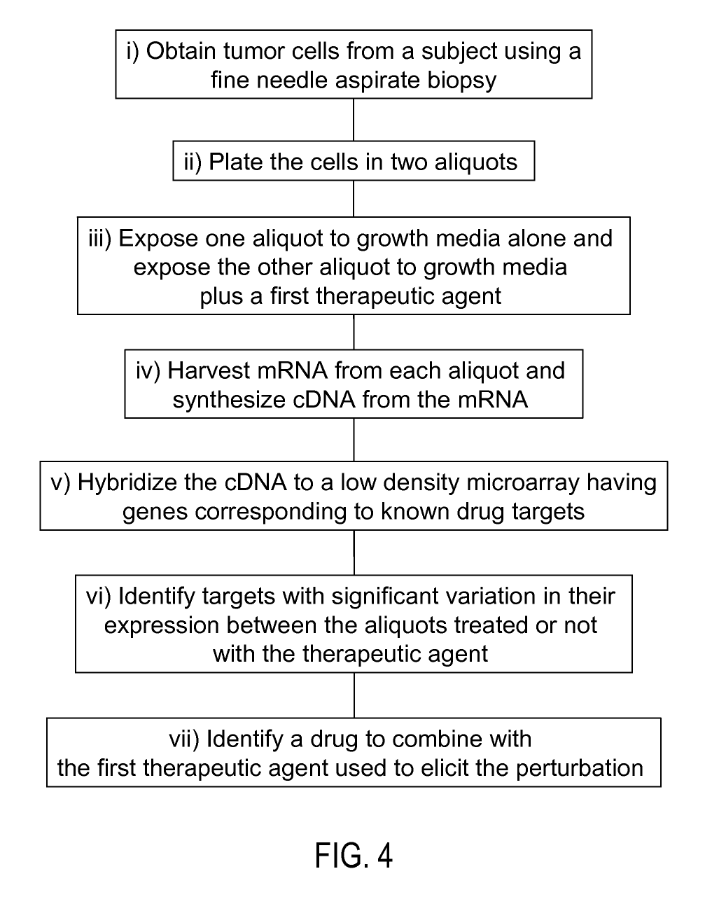

An example embodiment of the perturbability assay of the present invention is outlined in FIG. 4. In this example, the perturbability assay is performed as follows: i) obtaining tumor cells from a subject, e.g., by a fine needle aspiration biopsy; ii) plating of the cells in two aliquots; iii) exposure of one aliquot to growth media alone and exposure of the other aliquot to growth media plus a therapeutic agent, e.g., the standard agent for a given disease, to create a perturbation; iv) harvesting of mRNA from each aliquot and synthesizing cDNA from the mRNA; v) determining gene expression profiles using a microarray, e.g., a 384-well low-density microarray (LDMA), that may be customized, e.g., for a given disease; vi) bioinformatic analysis of the mRNA expression profiles to identify targets with significant variation in their expression between the aliquots treated or not with the therapeutic agent; and vii) identification of a rational drug to combine with the first therapeutic agent used to elicit the perturbation.

In some embodiments, the assay is customized to any biological sample, e.g. a sample from any of the tumor types disclosed herein. In some embodiments, the assay is based on acquiring a subject sample, e.g., a fine needle aspiration of a neoplastic lesion or other sample as described herein. In some embodiments, the sample is derived from a paraffin block. In some embodiments, the samples are frozen. In some embodiments, the samples are fresh. Where multiple gene targets are assayed, gene targets can be assayed that are known to be relevant in a specific disease. In some embodiments, a panel of gene targets is chosen that is useful for any disease. In some embodiments, the nucleic acids assayed with a low density microarray represent targets for which an inhibiting drug is known. The microarray can include various gene sets as are applicable, e.g., 45 to 180 genes. The gene targets can be chosen on the basis of the availability of agents and known combinations for each clinical scenario.

In some embodiments, a high-throughput, fully quantitative mRNA assessment is done using low-density microarrays (LDMA) comparing the gene expression of a set of 45 to 180 genes representing targets for which an inhibiting drug exists in both unexposed and exposed samples. Alternate techniques such as RT-PCR could be used instead of LDMA to determine gene expression levels. In some embodiments, the genes chosen to be assayed include targets for a particular disease or family of diseases. In some embodiments, the genes chosen to be assayed include members of a gene set, e.g., a biological pathway, that is deregulated in a particular disease or family of diseases. In some embodiments, the genes chosen to be assayed are associated with known drug targets. Deregulated gene sets include those that are differentially expressed in one cell versus another, e.g. in a tumor cell versus a normal cell, or between two tumor cells, etc. Differential expression includes both overexpression (upregulation) and underexpression (downregulation). In a deregulated pathway, not all genes need be differentially expressed, but only a sufficient number for the computational methods disclosed herein to detect differences. In some embodiments, the genes chosen to be assayed can include the genes listed in TABLE 8. In some embodiments, microarrays comprising hundreds to thousands of genes are used to measure gene expression, as described herein. In some embodiments, the expression levels are normalized by subtracting from each gene the expression levels of housekeeping genes determined in the same experiment. The expression of the housekeeping genes should be minimally affected by cellular perturbation such as contact with a therapeutic agent. Useful housekeeping genes include UBC, HPRT and SDHA.

In still other embodiments, the expression profiles are used to perform gene set analysis as described herein. In some embodiments, gene set analysis is used to guide selection of the second therapeutic agent. For example, the microarray data can be used to determine gene set expression profiles, also referred to as gene set expression signatures. In some embodiments, gene set expression profiles of the exposed and unexposed cells are compared to determine gene sets that are deregulated in the exposed cells after treatment with the first therapeutic agent. In some embodiments, the second therapeutic agent is chosen to targets these deregulated gene sets. In some embodiments, the gene set expression profiles are analyzed using Gene Set Enrichment Analysis (GSEA). Numerous alternate bioinformatics approaches can be used to analyze gene set expression profiles, as described herein. In some embodiments, gene sets correspond to biological pathways, such as metabolic pathways, developmental pathways, signal-transduction pathways, and genetic regulatory circuits. In some embodiments, gene sets are selected as defined by the KEGG database. In some embodiments, gene sets are selected as defined by the GO ontologies. Gene sets can further include groups of genes that share, e.g., common biological function, chromosomal location, or regulation. Any gene set comprising groups of genes can be used.

The target cell for the assay can come from any number or sources, as described herein. In some embodiments, the target cell is a tumor cell. The target cell can be derived from any anatomical origin or any cancer type, as described herein. The tumor cell can be extracted from a subject as described herein, e.g., using a fine needle aspirate biopsy.

One of skill in the art will appreciate that the method can be used with any therapeutic agent, as described herein.

### 3. Molecular Biology Techniques

In various embodiments, methods of the invention comprise using one or more techniques including techniques based in biology (including recombinant techniques), microbiology, cell biology, biochemistry, nucleic acid chemistry, and immunology, which are well known to those skilled in the art. Such techniques are explained fully in the literature, such as, Molecular Cloning: A Laboratory Manual, second edition (Sambrook et al., 1989) and Molecular Cloning: A Laboratory Manual, third edition (Sambrook and Russel, 2001); Current Protocols in Molecular Biology (F. M. Ausubel et al., eds., 1987, including supplements through 2001); PCR: The Polymerase Chain Reaction, (Mullis et al., eds., 1994); Harlow and Lane (1988) Antibodies, A Laboratory Manual, Cold Spring Harbor Publications, New York; Harlow and Lane (1999) Using Antibodies: A Laboratory Manual Cold Spring Harbor Laboratory Press, Cold Spring Harbor, N.Y., Beaucage et al. eds., Current Protocols in Nucleic Acid Chemistry John Wiley & Sons, Inc., New York, 2000) and Casarett and Doull's Toxicology The Basic Science of Poisons, C. Klaassen, ed., 6th edition (2001).

### 4. Biological Samples

In various embodiments, methods and compositions of the invention comprise assessing a biological sample. For example, the invention may be practiced with the use of many types of biological samples, e.g., samples containing cells. In addition, any nucleic acid containing sample which may be assayed for gene expression levels can be used in the practice of the invention. In some embodiments, a sample of the invention is suspected or known to contain tumor cells. Alternatively, a sample of the invention may be a “tumor sample” or “tumor containing sample” or “tumor cell containing sample” of tissue or fluid isolated from an individual suspected of being afflicted with, or at risk of developing, cancer. Examples of tumor samples include tumor cells, xenografts, and tumor samples, such as a resected tumor, tumor biopsy, and archived tumor sections. Further examples of samples for use with the invention include a clinical sample, such as, but not limited to, a fixed sample, a fresh sample, or a frozen sample. In some embodiments, the sample comprise paraffin block samples. The sample may be an aspirate, a cytological sample (including blood or other bodily fluid), or a tissue specimen, which includes at least some information regarding the in situ context of cells in the specimen, so long as appropriate cells or nucleic acids are available for determination of gene expression levels. In some embodiments, the samples are extracted from a subject using a fine needle aspirate biopsy technique.

The cell sample may be one of cancer cells from various cellular origins enriched from the blood of a subject, such as by use of labeled antibodies against cell surface markers followed by fluorescence activated cell sorting (FACS). In some embodiments, the antibodies are labeled to permit their detection after binding to the gene product. In some embodiments, microbeads are used to capture cells.

In some embodiments, the sample for the assay is provided by procurement of lesion tissue from a subject, including, e.g., radiologic assessment (ultrasound or computed tomography [CT]). These procedures have minimal comorbidities, can take a short time (e.g., approximately 30 minutes), and can be done in an outpatient setting. Sample processing can be performed by any standard molecular biology research laboratory.

In some embodiments, fixed samples are used. Fixed samples include those that are fixed with formalin or formaldehyde (including FFPE samples), with Boudin's, glutaldehyde, acetone, alcohols, or any other fixative, such as those used to fix cell or tissue samples for immunohistochemistry (IHC). Other examples include fixatives that precipitate cell associated nucleic acids and proteins. In some embodiments, samples are contained within paraffin blocks. Given possible complications in handling frozen tissue specimens, such as the need to maintain its frozen state, the invention may be practiced with non-frozen samples, such as fixed samples, fresh samples, including cells from blood or other bodily fluid or tissue, and minimally treated samples. In some applications of the invention, the sample has not been classified using standard pathology techniques, such as, but not limited to, immunohistochemistry based assays.

(a) Sample Origin

In some embodiments, methods of the present invention comprise selecting one or more therapeutic agents for treating diseases derived from any anatomical origin.

In some embodiments of the invention, the sample is classified as containing a cell of a type selected from the following types and subsets thereof: adrenal, brain, breast, carcinoid-intestine, cervix (squamous cell), cholangiocarcinoma, endometrium, germ-cell, GIST (gastrointestinal stromal tumor), kidney, leiomyosarcoma, liver, lung (adenocarcinoma, large cell), lung (small cell), lung (squamous), lymphoma (B cell), Lymphoma (Hodgkins), meningioma, mesothelioma, osteosarcoma, ovary (clear cell), ovary (serous cell), pancreas, prostate, skin (basal cell), skin (melanoma), small and large bowel; soft tissue (liposarcoma); soft tissue (MFH or Malignant Fibrous Histiocytoma), soft tissue (Sarcoma-synovial), testis (seminoma), thyroid (follicular-papillary), thyroid (medullary carcinoma), and urinary bladder. In some embodiments, the sample comprises a tumor cell.

In further embodiments of the invention, the sample is classified as containing a tumor cell of a type selected from the following types and subsets thereof: adrenal gland, brain, breast, carcinoid-intestine, cervix-adenocarcinoma, cervix-squamous, endometrium, gall bladder, germ cell-ovary, GIST, kidney, leiomyosarcoma, liver, lung-adenocarcinoma-large cell, lung-small cell, lung-squamous, lymphoma-B cell, lymphoma-Hodgkin's, lymphoma-T cell, meningioma, mesothelioma, osteosarcoma, ovary-clear cell, ovary-serous, pancreas, prostate, skin-basal cell, skin-melanoma, skin-squamous, small and large bowel, soft tissue-liposarcoma, soft tissue-MFH, soft tissue-sarcoma-synovial, stomach-adenocarcinoma, testis-other (or non-seminoma), testis-seminoma, thyroid-follicular-papillary, thyroid-medullary, and urinary bladder.

In some embodiments of the invention, the sample is classified as containing a tumor cell of a type selected from the following types and subsets thereof: Adenocarcinoma of Breast, Adenocarcinoma of Cervix, Adenocarcinoma of Esophagus, Adenocarcinoma of Gall Bladder, Adenocarcinoma of Lung, Adenocarcinoma of Pancreas, Adenocarcinoma of Small-Large Bowel, Adenocarcinoma of Stomach, Astrocytoma, Basal Cell Carcinoma of Skin, Cholangiocarcinoma of Liver, Clear Cell Adenocarcinoma of Ovary, Diffuse Large B-Cell Lymphoma, Embryonal Carcinoma of Testes, Endometrioid Carcinoma of Uterus, Ewings Sarcoma, Follicular Carcinoma of Thyroid, Gastrointestinal Stromal Tumor, Germ Cell Tumor of Ovary, Germ Cell Tumor of Testes, Glioblastoma Multiforme, Hepatocellular Carcinoma of Liver, Hodgkin's Lymphoma, Large Cell Carcinoma of Lung, Leiomyosarcoma, Liposarcoma, Lobular Carcinoma of Breast, Malignant Fibrous Histiocytoma, Medulary Carcinoma of Thyroid, Melanoma, Meningioma, Mesothelioma of Lung, Mucinous Adenocarcinoma of Ovary, Myofibrosarcoma, Neuroendocrine Tumor of Bowel, Oligodendroglioma, Osteosarcoma, Papillary Carcinoma of Thyroid, Pheochromocytoma, Renal Cell Carcinoma of Kidney, Rhabdomyosarcoma, Seminoma of Testes, Serous Adenocarcinoma of Ovary, Small Cell Carcinoma of Lung, Squamous Cell Carcinoma of Cervix, Squamous Cell Carcinoma of Esophagus, Squamous Cell Carcinoma of Larynx, Squamous Cell Carcinoma of Lung, Squamous Cell Carcinoma of Skin, Synovial Sarcoma, T-Cell Lymphoma, and Transitional Cell Carcinoma of Bladder.

(b) Cancers

In various embodiments, methods of the invention comprise selecting one or more therapeutic agents for any cancer. In one embodiment, the invention provides a method of improving treatments for breast cancer such as a ductal carcinoma in duct tissue in a mammary gland, medullary carcinomas, colloid carcinomas, tubular carcinomas, and inflammatory breast cancer. In further embodiments, methods of the invention comprise improving treatments for ovarian cancer, including epithelial ovarian tumors such as adenocarcinoma in the ovary and an adenocarcinoma that has migrated from the ovary into the abdominal cavity. Similarly the invention provides a method of improving treatments for cervical cancer such as adenocarcinoma in the cervix epithelial including squamous cell carcinoma and adenocarcinomas.

In additional embodiments, methods of the invention comprise selecting one or more therapeutic agents to treat prostate cancer, such as a prostate cancer selected from the following: an adenocarcinoma or an adenocarcinoma that has migrated to the bone; treatments for pancreatic cancer such as epitheliod carcinoma in the pancreatic duct tissue and an adenocarcinoma in a pancreatic duct; treatments for bladder cancer such as a transitional cell carcinoma in urinary bladder, urothelial carcinomas (transitional cell carcinomas), tumors in the urothelial cells that line the bladder, squamous cell carcinomas, adenocarcinomas, and small cell cancers. Similarly, the invention provides methods of improving treatments for acute myeloid leukemia (AML), preferably acute promyelocytic leukemia in peripheral blood. There are other types of leukemia's that can also be treated by the methods provided by the invention including but not limited to, Acute Lymphocytic Leukemia, Acute Myeloid Leukemia, Chronic Lymphocytic Leukemia, Chronic Myeloid Leukemia, Hairy Cell Leukemia, Myelodysplasia, and Myeloproliferative Disorders. Similarly the invention provides methods for improving treatments for lung cancer such as non-small cell lung cancer (NSCLC), which is divided into squamous cell carcinomas, adenocarcinomas, and large cell undifferentiated carcinomas, and small cell lung cancer. Similarly the invention provides methods for improving treatments for skin cancer such as basal cell carcinoma, melanoma, squamous cell carcinoma and actinic keratosis, which is a skin condition that sometimes develops into squamous cell carcinoma; treatments for eye retinoblastoma; improving treatments for intraocular (eye) melanoma; improving treatments for primary liver cancer (cancer that begins in the liver); improving treatments for kidney cancer.

In another aspect, the invention provides methods for improving treatments for thyroid cancer such as papillary, follicular, medullary and anaplastic; improving treatments for AIDS-related lymphoma such as diffuse large B-cell lymphoma, B-cell immunoblastic lymphoma and small non-cleaved cell lymphoma; improving treatments for Kaposi's sarcoma; improving treatments for viral-induced cancers. The major virus-malignancy systems include hepatitis B virus (HBV), hepatitis C virus (HCV), and hepatocellular carcinoma; human lymphotrophic virus-type 1 (HTLV-1) and adult T-cell leukemia/lymphoma; and human papilloma virus (HPV) and cervical cancer. Similarly the invention provides methods for improving treatments for central nervous system cancers such as primary brain tumor, which includes gliomas (astrocytoma, anaplastic astrocytoma, or glioblastoma multiforme), Oligodendroglioma, Ependymoma, Meningioma, Lymphoma, Schwannoma, and Medulloblastoma. Similarly the invention provides methods for improving treatments for peripheral nervous system (PNS) cancers such as acoustic neuromas and malignant peripheral nerve sheath tumor (MPNST) including neurofibromas and schwannomas. Other types of PNS cancers include but not limited to, malignant fibrous cytoma, malignant fibrous histiocytoma, malignant meningioma, malignant mesothelioma, and malignant mixed Müllerian tumor. Similarly the invention provides methods for improving treatments for oral cavity and oropharyngeal cancer. These include cancers such as, hypopharyngeal cancer, laryngeal cancer, nasopharyngeal cancer, oropharyngeal cancer, and the like. Similarly the invention provides methods for improving treatments for stomach cancer such as lymphomas, gastric stromal tumors, and carcinoid tumors. Similarly the invention provides methods for improving treatments for testicular cancer such as germ cell tumors (GCTs), which include seminomas and nonseminomas; and gonadal stromal tumors, which include Leydig cell tumors and Sertoli cell tumors. Similarly the invention provides methods for improving treatments for testicular cancer such as thymus cancer, such as to thymomas, thymic carcinomas, Hodgkin disease, non-Hodgkin lymphomas carcinoids or carcinoid tumors.

In some embodiments, the cancer comprises Acute Lymphoblastic Leukemia. In other embodiments, the cancer comprises Acute Myeloid Leukemia. In other embodiments, the cancer comprises Adrenocortical Carcinoma. In other embodiments, the cancer comprises an AIDS-Related Cancer. In other embodiments, the cancer comprises AIDS-Related Lymphoma. In other embodiments, the cancer comprises Anal Cancer. In other embodiments, the cancer comprises Appendix Cancer. In other embodiments, the cancer comprises Childhood Cerebellar Astrocytoma. In other embodiments, the cancer comprises Childhood Cerebral Astrocytoma. In other embodiments, the cancer comprises a Central Nervous System Atypical Teratoid/Rhabdoid Tumor. In other embodiments, the cancer comprises Basal Cell Carcinoma, or other Skin Cancer (Nonmelanoma). In other embodiments, the cancer comprises Extrahepatic Bile Duct Cancer. In other embodiments, the cancer comprises Bladder Cancer. In other embodiments, the cancer comprises Bone Cancer, such as Osteosarcoma or Malignant Fibrous Histiocytoma. In other embodiments, the cancer comprises Brain Stem Glioma. In other embodiments, the cancer comprises an Adult Brain Tumor. In other embodiments, the cancer comprises Brain Tumor, Central Nervous System Atypical Teratoid/Rhabdoid Tumor, Childhood. In other embodiments, the cancer comprises a Brain Tumor comprising Cerebral Astrocytoma/Malignant Glioma. In other embodiments, the cancer comprises a Craniopharyngioma Brain Tumor. In other embodiments, the cancer comprises a Ependymoblastoma Brain Tumor. In other embodiments, the cancer comprises a Ependymoma Brain Tumor. In other embodiments, the cancer comprises a Medulloblastoma Brain Tumor. In other embodiments, the cancer comprises a Medulloepithelioma Brain Tumor. In other embodiments, the cancer comprises Brain Tumors including Pineal Parenchymal Tumors of Intermediate Differentiation. In other embodiments, the cancer comprises Brain Tumors including Supratentorial Primitive Neuroectodermal Tumors and Pineoblastoma. In other embodiments, the cancer comprises a Brain Tumor including Visual Pathway and Hypothalamic Glioma. In other embodiments, the cancer comprises Brain and Spinal Cord Tumors. In other embodiments, the cancer comprises Breast Cancer. In other embodiments, the cancer comprises Bronchial Tumors. In other embodiments, the cancer comprises Burkitt Lymphoma. In other embodiments, the cancer comprises Carcinoid Tumor. In other embodiments, the cancer comprises Gastrointestinal Carcinoid Tumor. In other embodiments, the cancer comprises Carcinoma of Unknown Primary Origin. In other embodiments, the cancer comprises Central Nervous System Atypical Teratoid/Rhabdoid Tumor. In other embodiments, the cancer comprises Central Nervous System Embryonal Tumors. In other embodiments, the cancer comprises Primary Central Nervous System Lymphoma. In other embodiments, the cancer comprises Cerebellar Astrocytoma. In other embodiments, the cancer comprises Cerebral Astrocytoma/Malignant Glioma. In other embodiments, the cancer comprises Cervical Cancer. In other embodiments, the cancer comprises Childhood Cancers. In other embodiments, the cancer comprises Chordoma. In other embodiments, the cancer comprises Chronic Lymphocytic Leukemia. In other embodiments, the cancer comprises Chronic Myelogenous Leukemia. In other embodiments, the cancer comprises Chronic Myeloproliferative Disorders. In other embodiments, the cancer comprises Colon Cancer. In other embodiments, the cancer comprises Colorectal Cancer. In other embodiments, the cancer comprises Craniopharyngioma. In other embodiments, the cancer comprises Cutaneous T-Cell Lymphoma, including Mycosis Fungoides and Sézary Syndrome. In other embodiments, the cancer comprises Central Nervous System Embryonal Tumors. In other embodiments, the cancer comprises Endometrial Cancer. In other embodiments, the cancer comprises Ependymoblastoma. In other embodiments, the cancer comprises Ependymoma. In other embodiments, the cancer comprises Esophageal Cancer. In other embodiments, the cancer comprises the Ewing Family of Tumors. In other embodiments, the cancer comprises Extracranial Germ Cell Tumor. In other embodiments, the cancer comprises Extragonadal Germ Cell Tumor. In other embodiments, the cancer comprises Extrahepatic Bile Duct Cancer. In other embodiments, the cancer comprises Intraocular Melanoma Eye Cancer. In other embodiments, the cancer comprises Retinoblastoma Eye Cancer. In other embodiments, the cancer comprises Gallbladder Cancer. In other embodiments, the cancer comprises Gastric (Stomach) Cancer. In other embodiments, the cancer comprises Gastrointestinal Carcinoid Tumor. In other embodiments, the cancer comprises Gastrointestinal Stromal Tumor (GIST). In other embodiments, the cancer comprises Gastrointestinal Stromal Cell Tumor. In other embodiments, the cancer comprises Extracranial Germ Cell Tumor. In other embodiments, the cancer comprises Extragonadal Germ Cell Tumor. In other embodiments, the cancer comprises Ovarian Germ Cell Tumor. In other embodiments, the cancer comprises Gestational Trophoblastic Tumor. In other embodiments, the cancer comprises Glioma. In other embodiments, the cancer comprises Brain Stem Glioma. In other embodiments, the cancer comprises Cerebral Astrocytoma Glioma. In other embodiments, the cancer comprises Visual Pathway or Hypothalamic Glioma. In other embodiments, the cancer comprises Hairy Cell Leukemia. In other embodiments, the cancer comprises Head and Neck Cancer. In other embodiments, the cancer comprises Hepatocellular (Liver) Cancer. In other embodiments, the cancer comprises Hodgkin Lymphoma. In other embodiments, the cancer comprises Hypopharyngeal Cancer. In other embodiments, the cancer comprises Intraocular Melanoma. In other embodiments, the cancer comprises Islet Cell Tumors (Endocrine Pancreas). In other embodiments, the cancer comprises Kaposi Sarcoma. In other embodiments, the cancer comprises Kidney (Renal Cell) Cancer. In other embodiments, the cancer comprises Laryngeal Cancer. In other embodiments, the cancer comprises Acute Lymphoblastic Leukemia. In other embodiments, the cancer comprises Acute Myeloid Leukemia. In other embodiments, the cancer comprises Chronic Lymphocytic Leukemia. In other embodiments, the cancer comprises Chronic Myelogenous Leukemia. In other embodiments, the cancer comprises Hairy Cell Leukemia. In other embodiments, the cancer comprises Lip Cancer. In other embodiments, the cancer comprises Oral Cavity Cancer. In other embodiments, the cancer comprises Primary Liver Cancer. In other embodiments, the cancer comprises Non-Small Cell Lung Cancer. In other embodiments, the cancer comprises Small Cell Lung Cancer. In other embodiments, the cancer comprises AIDS-Related Lymphoma. In other embodiments, the cancer comprises Burkitt Lymphoma. In other embodiments, the cancer comprises Cutaneous T-Cell Lymphoma. In other embodiments, the cancer comprises Mycosis Fungoides and Sézary Syndrome. In other embodiments, the cancer comprises Hodgkin Lymphoma. In other embodiments, the cancer comprises Non-Hodgkin Lymphoma. In other embodiments, the cancer comprises Primary Central Nervous System Lymphoma. In other embodiments, the cancer comprises Waldenström Macroglobulinemia. In other embodiments, the cancer comprises Malignant Fibrous Histiocytoma of Bone or Osteosarcoma. In other embodiments, the cancer comprises Medulloepithelioma. In other embodiments, the cancer comprises Melanoma. In other embodiments, the cancer comprises Intraocular (Eye) Melanoma. In other embodiments, the cancer comprises Merkel Cell Carcinoma. In other embodiments, the cancer comprises Mesothelioma. In other embodiments, the cancer comprises Metastatic Squamous Neck Cancer with Occult Primary. In other embodiments, the cancer comprises Mouth Cancer. In other embodiments, the cancer comprises Multiple Endocrine Neoplasia Syndrome. In other embodiments, the cancer comprises Multiple Myeloma/Plasma Cell Neoplasm. In other embodiments, the cancer comprises Mycosis Fungoides. In other embodiments, the cancer comprises Myelodysplastic Syndromes. In other embodiments, the cancer comprises Myelodysplastic or Myeloproliferative Diseases. In other embodiments, the cancer comprises Chronic Myelogenous Leukemia. In other embodiments, the cancer comprises Acute Myeloid Leukemia. In other embodiments, the cancer comprises Multiple Myeloma. In other embodiments, the cancer comprises Chronic Myeloproliferative Disorders. In other embodiments, the cancer comprises Nasal Cavity or Paranasal Sinus Cancer. In other embodiments, the cancer comprises Nasopharyngeal Cancer. In other embodiments, the cancer comprises Nasopharyngeal Cancer. In other embodiments, the cancer comprises Neuroblastoma. In other embodiments, the cancer comprises Non-Hodgkin Lymphoma. In other embodiments, the cancer comprises Non-Small Cell Lung Cancer. In other embodiments, the cancer comprises Oral Cancer. In other embodiments, the cancer comprises Oral Cavity Cancer. In other embodiments, the cancer comprises Oropharyngeal Cancer. In other embodiments, the cancer comprises Osteosarcoma. In other embodiments, the cancer comprises Malignant Fibrous Histiocytoma of Bone. In other embodiments, the cancer comprises Ovarian Cancer. In other embodiments, the cancer comprises Ovarian Epithelial Cancer. In other embodiments, the cancer comprises Ovarian Germ Cell Tumor. In other embodiments, the cancer comprises Ovarian Low Malignant Potential Tumor. In other embodiments, the cancer comprises Pancreatic Cancer. In other embodiments, the cancer comprises Islet Cell Tumor Pancreatic Cancer. In other embodiments, the cancer comprises Papillomatosis. In other embodiments, the cancer comprises Paranasal Sinus Cancer. In other embodiments, the cancer comprises Nasal Cavity Cancer. In other embodiments, the cancer comprises Parathyroid Cancer. In other embodiments, the cancer comprises Penile Cancer. In other embodiments, the cancer comprises Pharyngeal Cancer. In other embodiments, the cancer comprises Pheochromocytoma. In other embodiments, the cancer comprises Pineal Parenchymal Tumors of Intermediate Differentiation. In other embodiments, the cancer comprises Pineoblastoma or Supratentorial Primitive Neuroectodermal Tumors. In other embodiments, the cancer comprises Pituitary Tumor. In other embodiments, the cancer comprises Plasma Cell Neoplasm/Multiple Myeloma. In other embodiments, the cancer comprises Pleuropulmonary Blastoma. In other embodiments, the cancer comprises Primary Central Nervous System Lymphoma. In other embodiments, the cancer comprises Prostate Cancer. In other embodiments, the cancer comprises Rectal Cancer. In other embodiments, the cancer comprises Renal Cell (Kidney) Cancer. In other embodiments, the cancer comprises Renal Pelvis and Ureter, Transitional Cell Cancer. In other embodiments, the cancer comprises Respiratory Tract Carcinoma Involving the NUT Gene on Chromosome 15. In other embodiments, the cancer comprises Retinoblastoma. In other embodiments, the cancer comprises Rhabdomyosarcoma. In other embodiments, the cancer comprises Salivary Gland Cancer. In other embodiments, the cancer comprises Sarcoma of the Ewing Family of Tumors. In other embodiments, the cancer comprises Kaposi Sarcoma. In other embodiments, the cancer comprises Soft Tissue Sarcoma. In other embodiments, the cancer comprises Uterine Sarcoma. In other embodiments, the cancer comprises Sezary Syndrome. In other embodiments, the cancer comprises Nonmelanoma Skin Cancer. In other embodiments, the cancer comprises Melanoma Skin Cancer. In other embodiments, the cancer comprises Merkel Cell Skin Carcinoma. In other embodiments, the cancer comprises Small Cell Lung Cancer. In other embodiments, the cancer comprises Small Intestine Cancer. In other embodiments, the cancer comprises Squamous Cell Carcinoma, e.g., Nonmelanoma Skin Cancer. In other embodiments, the cancer comprises Metastatic Squamous Neck Cancer with Occult Primary. In other embodiments, the cancer comprises Stomach (Gastric) Cancer. In other embodiments, the cancer comprises Supratentorial Primitive Neuroectodermal Tumors. In other embodiments, the cancer comprises Cutaneous T-Cell Lymphoma, e.g., Mycosis Fungoides and Sezary Syndrome. In other embodiments, the cancer comprises Testicular Cancer. In other embodiments, the cancer comprises Throat Cancer. In other embodiments, the cancer comprises Thymoma or Thymic Carcinoma. In other embodiments, the cancer comprises Thyroid Cancer. In other embodiments, the cancer comprises Transitional Cell Cancer of the Renal Pelvis and Ureter. In other embodiments, the cancer comprises Gestational Trophoblastic Tumor. In other embodiments, the cancer comprises a Carcinoma of Unknown Primary Site. In other embodiments, the cancer comprises an Unusual Cancer of Childhood. In other embodiments, the cancer comprises Ureter and Renal Pelvis Transitional Cell Cancer. In other embodiments, the cancer comprises Urethral Cancer. In other embodiments, the cancer comprises Endometrial Uterine Cancer. In other embodiments, the cancer comprises Uterine Sarcoma. In other embodiments, the cancer comprises Vaginal Cancer. In other embodiments, the cancer comprises Visual Pathway and Hypothalamic Glioma. In other embodiments, the cancer comprises Vulvar Cancer. In other embodiments, the cancer comprises Waldenström Macroglobulinemia. In other embodiments, the cancer comprises Wilms Tumor. In other embodiments, the cancer comprises Women's Cancers.

### 5. Therapeutic Agents

In various embodiments, the present invention is used for selecting any therapeutic known to treat a cell, e.g., a cancer cell. In some embodiments, the methods of the present invention can identify one or more therapeutic agents that are more likely to be effective in that particular biologic environment. The methods enhance the individualization of that given subject's therapy and the chances of a successful intervention.

Examples of therapeutic agents include drugs, chemical compounds, small molecules, nucleic acids, such as siRNA, microRNA or antisense therapies, biologic agents, such as antibodies, cytokines or proteins, polysaccharides, peptides, radiation, vaccines or multimodality approaches. In some embodiments, the method of the present invention is used to select therapeutic agents comprising drugs. In other embodiments, the method of the present invention is used to select therapeutic agents comprising small molecules. In other embodiments, the method of the present invention is used to select therapeutic agents comprising nucleic acids, such as siRNA, microRNA or antisense therapies. In other embodiments, the method of the present invention is used to select therapeutic agents comprising biologic agents. In other embodiments, the method of the present invention is used to select therapeutic agents comprising biological agents that are antibodies. In other embodiments, the method of the present invention is used to select therapeutic agents comprising biological agents that are cytokines. In other embodiments, the method of the present invention is used to select therapeutic agents comprising biological agents that are proteins. In other embodiments, the method of the present invention is used to select therapeutic agents comprising biological agents that are polysaccharides. In other embodiments, the method of the present invention is used to select therapeutic agents comprising biological agents that are peptides. In other embodiments, the method of the present invention is used to select therapeutic agents comprising radiation therapy. In other embodiments, the method of the present invention is used to select therapeutic agents comprising vaccines. Such vaccines can comprise cancer vaccines.

Therapeutic agents comprise chemotherapeutic agents, which refers to all chemical compounds that are effective in inhibiting tumor growth. Examples of chemotherapeutic agents include alkylating agents; for example, nitrogen mustards, ethyleneimine compounds and alkyl sulphonates; antimetabolites; for example, folic acid, purine or pyrimidine antagonists; mitotic inhibitors; for example, vinca alkaloids and derivatives of podophyllotoxin, cytotoxic antibiotics, compounds that damage or interfere with DNA expression, and growth factor receptor antagonists. In addition, chemotherapeutic agents include cytotoxic agents (as defined herein), antibodies, biological molecules and small molecules. A cytotoxic agent includes a substance that inhibits or prevents the expression activity of cells, function of cells and/or causes destruction of cells. These include radioactive isotopes, chemotherapeutic agents, and toxins such as small molecule toxins or enzymatically active toxins of bacterial, fungal, plant or animal origin, including fragments and/or variants thereof. Examples of cytotoxic agents include, but are not limited to auristatins, auromycins, maytansinoids, yttrium, bismuth, ricin, ricin A-chain, combrestatin, duocarmycins, dolostatins, doxorubicin, daunorubicin, taxol, cisplatin, cc1065, ethidium bromide, mitomycin, etoposide, tenoposide, vincristine, vinblastine, colchicine, dihydroxy anthracin dione, actinomycin, diphtheria toxin, Pseudomonas exotoxin (PE) A, PE40, abrin, abrin A chain, modeccin A chain, alpha-sarcin, gelonin, mitogellin, restrictocin, phenomycin, enomycin, curicin, crotin, calicheamicin, Sapaonaria officinalis inhibitor, and glucocorticoid and other chemotherapeutic agents, as well as radioisotopes. Antibodies may also be conjugated to an anti-cancer pro-drug activating enzyme capable of converting the pro-drug to its active form. In some embodiments, therapeutic agents comprise inhibitors against known gene products.

Those of skill in the art will appreciate that any therapeutic agent can be selected using the present invention. A comprehensive listing of drugs and other therapeutic agents used to treat cancer or conditions related to cancer is maintained by the National Cancer Institute (NCI) of the U.S. National Institutes of Health. See www.cancer.gov/drugdictionary/.

In other embodiments, methods of the invention comprise selecting multiple therapeutic agents. For example, a number of drugs may be known for treating the reference cell that is determined to be most identical to the target cell. In that case, a plurality of therapeutics agent can be selected for treatment. Such combination therapy can be useful if, e.g., the plurality of therapeutic agents are known to affect alternate cellular functions. Therapeutic regimens comprising treatment with multiple drugs or other therapies are well known to those of skill in the art. See, e.g. Henkin et al., U.S. Patent Application 20060258597; Dugan, U.S. Pat. No. 7,294,332; Masferrer et al. U.S. Pat. No. 7,320,996; Stein et al., U.S. Pat. No. 7,351,729; Besterman et al., U.S. Pat. No. 6,953,783; Wilson U.S. Pat. No. 6,667,337; Baldwin et al., U.S. Pat. No. 6,831,057; Schwendner et al., U.S. Pat. No. 6,822,001; and McKearn et al., U.S. Pat. No. 6,858,598.

Those of skill in the art will appreciate that the present invention can be used to select therapeutic agents that are related to or structurally similar to agents used to create the reference panel, even if data for the selected therapeutic agent is not found directly within the reference database. For example, the reference panel may comprise data for rapamycin, a drug with a potent immunosuppressive and antiproliferative properties. When the method identifies a connected reference cell with known sensitivity to rapamycin, the methods of the present invention can select temsirolimus, an ester analog of rapamycin with improved pharmaceutical properties and aqueous solubility. In some embodiments, a subject with a tumor that is connected to rapamycin sensitive reference cells is treated with temsirolimus according to the present invention. In other embodiments, a subject with a tumor that is connected to docetaxel is treated with paclitaxel.

The present invention also allows selection of therapeutic agents other than those used to derive the reference database of drug sensitivity. For example, the reference database can have drug sensitivity data for a first drug but a structurally related compound with improved metabolic stability and efficacy is available. In some embodiments, the related compound is selected when the target sample is connected to a reference cell sensitive to the first drug without departing from the present invention. Structurally related therapeutic agents include, without limitation, analogs, homologs, derivatives, isomers, mimetics, metabolic derivatives, secondary metabolites, esters, or salt forms. Analogs include compounds with substituted atoms or functional groups, transition state analogs or similar structure. Isomers include, without limitation, stereoisomers, enantiomers, geometrical isomers, cis-trans isomers, conformers, rotamers, tautomers, topoisomers or constitutional (structural) isomers. As described herein, a structurally related compound could be a drug modified to improve pharmacological properties or processability. For a biological therapeutic agent, this could comprise a related peptide or immunotoxin. For example, the reference panel might include susceptibility data for a monoclonal antibody. The present invention might then be used to select a related therapeutic agent such as the monoclonal conjugated to one or more toxic agents. Such antibody drug conjugates are well known in the art. See, e.g., U.S. patent application Ser. No. 11/735,376, filed Apr. 13, 2007 and entitled “Anti-Cd70 Antibody-Drug Conjugates and Their Use for the Treatment of Cancer and Immune Disorders.” Alternately, the present invention can be used to select a peptide mimetic.

### 6. Computer Systems and Data Storage

In some embodiments, computer systems are used to perform a variety of logic operations of the present invention. The computer systems can include one or more computers, databases, memory systems, and system outputs (e.g., a computer screen or printer). In some embodiments, computer executable logic or program code is stored in a storage medium, loaded into and/or executed by a computer, or transmitted over some transmission medium, such as over electrical wiring or cabling, through fiber optics, or via electromagnetic radiation, e.g., wirelessly. When implemented on a general-purpose microprocessor, the computer executable logic can configure the microprocessor to create specific logic circuits. In some embodiments, multiple computer systems are used. In one embodiment, a patient or organization can provide target cell data either by uploading a tumor gene expression profile on a secure server (meeting industry requirements for security) or by sending the information in a high-density portable form (such as CDROM, DVD). The data can then be analyzed at a remote location.

In some embodiments, the computer system comprises a computer readable medium, e.g., floppy diskettes, CD-ROMs, hard drives, flash memory, tape, or other digital storage medium, with a program code comprising one or more sets of instructions for performing a variety of logic operations. In some embodiments, a computer system is used to analyze gene expression data and construct expression profiles. In some embodiments, a computer system is used to perform GSEA or other gene set analysis algorithms. In some embodiments, a computer system is used to determine gene expression profiles or gene set expression profiles. In some embodiments, a computer system is used to compare relevant biological characteristics of the target cell to the reference database, e.g., by correlating or classifying an expression profile of the target cell to the reference database. In some embodiments, a computer system is used to identify the most similar reference cell to the target cell, e.g., according to the comparison of biological characteristics. In some embodiments, a computer system is used to select appropriate therapeutic agents from a reference database. In some embodiments, a computer is used to compare the response of cell to a perturbation, e.g., contacting the cell with a therapeutic agent. In some embodiments, a computer is used to determine and compare gene expression profiles from cells that are contacted or not with the agent. In some embodiments, a computer is used to determine and compare gene set expression profiles from cells that are contacted or not contacted with the agent. In some embodiments, a computer is used to predict drug response using methods of the invention described herein.

In some embodiments, a computer system comprises computer executable logic for: a) determining a gene set expression profile for two or more genes in a target cell; b) comparing the gene set expression profile of the target cell to one or more gene set expression profiles of a panel of reference cells, wherein the panel comprises cells from more than two different cell types; c) identifying a reference cell from the panel that has the most similar gene set expression profile to the target cell according to the comparison in step b); and d) selecting a therapeutic agent known for treating a condition in the reference cell identified in step c). In other embodiments, a computer system comprises computer executable logic for: a) determining a response of a target cell to a first therapeutic agent using one or more expression profiles; and b) selecting a second therapeutic agent based on the response of the target cell to the first therapeutic agent.

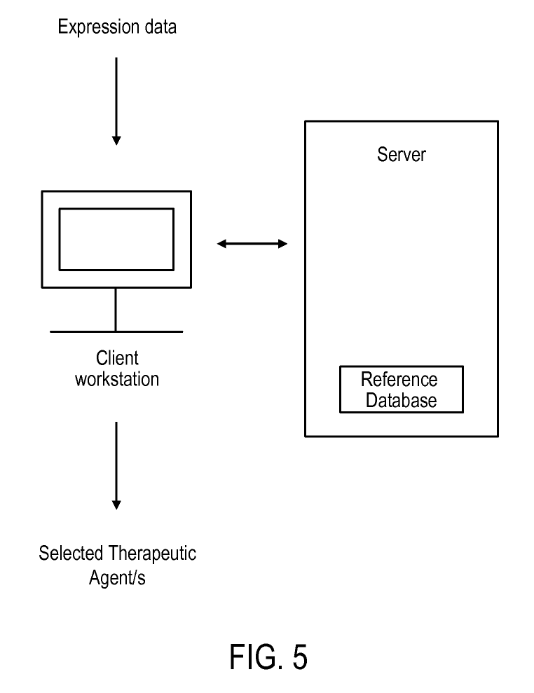

A reference database can be stored on a digital storage medium, e.g., floppy diskettes, CD-ROMs, hard drives, flash memory, tape, or other digital storage medium. In some embodiments, a reference database comprises relevant biological characteristics, e.g., gene expression profiles or gene set expression profiles, linked to therapeutic agent susceptibility data. The reference database can be stored locally or remotely with respect to the computer system used to perform logic operations. FIG. 5 illustrates an embodiment wherein the logic operations are performed on a client workstation computer and the reference database is stored on a server in networked communication with the client workstation.

### 7. Kits

In some embodiments, the various methods of the present invention are sold to end users in the form of a kit. In some embodiments, a kit includes reagents necessary to assay a target sample for use in the present invention. In some embodiments, a kit comprises materials useful for collecting a sample and sending the sample to a remote laboratory for analysis. In some embodiments, the kits comprise computer-related medium containing algorithms for use in the present invention. In some embodiments, the kit comprises computer-related medium containing a reference database for use in the present invention. In some embodiments, the kits of the present invention comprise one or more of these items. In some embodiments, the kits include low density microarrays and corresponding probe sets for performing a perturbability assay according to the present invention. In some embodiments, standard molecular biology reagents, e.g., collection tubes, buffers and enzymes, are included with a kit of the present invention. In some embodiments, the end user supplies some of the necessary reagents and materials. In some embodiments, the kits comprise kits materials or agents used to separate cells of various types on the basis of their phenotypes, e.g., microbeads.

### EXAMPLES

### Example 1

### Gene Expression and Gene Set Enrichment Analysis

### 1. Microarray Analysis

Tumor cell lines and baseline tumors from xenografts and direct patient samples were profiled using Affymetrix U133 Plus 2.0 gene arrays. This gene array has about 54,000 probes comprising about 20,000 genes. Sample preparation and processing procedure was performed as described in the Affymetrix GeneChip® Expression Analysis Manual (Affymetrix Inc., Santa Clara, Calif.). Gene expression levels were converted to a rank-based matrix and standardized (mean=0, standard deviation=1) for each microarray. Probes from Affymetrix HG-U133 Plus2.0 gene array were mapped to the HG-U133A and HG-U133B probes based on the probe set identifiers.

### 2. Gene Set Enrichment Analysis

Gene Set analysis was performed using the Gene Set Enrichment Analysis (GSEA) software Version 2.0.1 obtained from the Broad Institute (http://www.broad.mit.edu/gsea). GSEA methodology is described in Subramanian A, Tamayo P, Mootha V K, et al: Gene set enrichment analysis: A knowledge-based approach for interpreting genome-wide expression profiles. Proceedings of the National Academy of Sciences 102:15545-15550, 2005. Genes represented by more than one probe were collapsed using the Collapse Probes utility to the probe with the maximum value. Gene set permutations were performed 500 times for each analysis and the gene set, e.g., pathway, list is sorted by the Normalized Enrichment Score (NES).

### Example 2

### Xenograft Treatment

### 1. Establishment of Xenografts

Four- to six-week-old female athymic (nu/nu) mice were purchased from Harlan (Harlan Laboratories, Washington, D.C.). The research protocol was approved by the Johns Hopkins University Animal Use and Care Committee and animals were maintained in accordance to guidelines of the American Association of Laboratory Animal Care. Xenografts obtained from F1 mice were excised and cut into small ˜3×3×3 mm fragments and then implanted subcutaneously in a group of five to six mice for each patient, with two small fragments in each mouse (F2) as described above for the original carcinoma. Half of the rest of the carcinoma was cryopreserved in liquid nitrogen and the other half is processed for biological studies. When the carcinoma reached a size of 1,500 mm³, they were excised, cut into ˜3×3×3 mm fragments, and transplanted to the final cohort of mice to be treated with the drugs (F3 and successive passages). Further details of the establishment of the direct-patient pancreatic cancer xenografts are described in Rubio-Viqueira B, Jimeno A, Cusatis G, et al. An In vivo Platform for Translational Drug Development in Pancreatic Cancer. Clin Cancer Res 2006 12:4652-61.

### 2. Drugs

Gemcitabine (Eli Lilly, Indianapolis, Ind.) was dissolved in saline. In some embodiments, docetaxel (Sanofi-Aventis, Bridgewater, N.J.) was dissolved in 50% ethanol and 50% DMSO. In other embodiments, docetaxel (Sanofi-Aventis, Bridgewater, N.J.) was dissolved in ethanol/polysorbate 80 as a stock solution and diluted 10-fold in 5% glucose for in vivo studies. Temsirolimus (Wyeth Research, Colleville, Pa.) was dissolved in 10% ethanol, 10% pluronic, and 80% PBS. All drugs were freshly prepared and used at an injection volume of 0.2 mL/20 g body weight. Drug doses and treatment schedules were obtained from published studies.

### 3. Treatment Protocol

Xenografts from this second mouse-to-mouse passage (F3) were allowed to grow to a size of ˜200 mm³, at which time five-to-six mice were randomly assigned to control and treatment groups. For gemcitabine treatment group, mice were treated with 100 mg/kg twice a week via intraperitoneal injection. For docetaxel treatment group, mice were treated with 20 mg/kg once a week via intraperitoneal injection. For temsirolimus treatment group, mice were treated with 20 mg/kg/day via intraperitoneal injection. Mice were treated for 21-28 days, monitored daily for signs of toxicity, and were weighed thrice a week. Tumor size was evaluated thrice a week by caliper measurements using the following formula: tumor volume=[length×width²]/2 as reported by van de Vijver M J, He Y D, van't Veer L J, et al. A Gene-Expression Signature as a Predictor of Survival in Breast Cancer. N Engl J Med 2002 347:1999-2009.

### 4. Relative Tumor Growth Inhibition

Relative tumor growth index (TGI) was calculated as TGI=(Tᵢ−T₀/Cᵢ−C₀) if Tᵢ>T₀ (indicates tumor growth inhibition) and TGI=(Tᵢ−T₀) if Tᵢ≦T₀ (indicates tumor regression). T and C represent tumor size in the treated and control group at the last of experiment, respectively; T₀ and C₀ represent tumor size in the treated and control group at the start of treatment, respectively.

### 5. Statistical Analyses

Statistical analysis was performed using software SPSS version 16 (SPSS Inc., Chicago, Ill.). P-values <0.05 were regarded as significant. Disease free survival (DFS) was analyzed using the Kaplan-Meier method and compared with the log-rank test.

### Example 3

### GS-CMAP for TP53 Mutational Status

In one example, pathway-expression signatures were used to compare oncogene TP53 mutants versus TP53 wild-type in the NCI-60 panel. The TP53 mutational status of the NCI-60 panel was obtained from the Cancer Genome Project of human cancer cell lines database. See Ikediobi O N, Davies H, Bignell G, et al. Mutation analysis of 24 known cancer genes in the NCI-60 cell line set. Mol Cancer Ther 5:2606-12 (2006). 44 of the 60 cell lines have at least one mutation as recorded in the database and were considered mutants. The remaining 16 cell lines have no TP53 mutations and were considered wild-type. TABLE 4 lists the TP53 mutational status for the NCI60 cell lines.

TABLE 4
TP53 mutational status of the NCI-60 cell lines.

| | |
|---|---|
| TP53 wild-type | TP53 mutants |

| | | | |
|---|---|---|---|
| NSCLC3 | NSCLC1 | BC6 | RC8 |
| NSCLC5 | NSCLC2 | BC7 | ME3 |
| NSCLC7 | NSCLC4 | BC8 | ME5 |
| CCS | NSCLC6 | OC1 | ME6 |
| BC1 | NSCLC8 | OC2 | PC1 |
| OC3 | NSCLC9 | OC4 | PC2 |
| LE6 | CC1 | OC5 | CNS1 |
| RC1 | CC2 | OC6 | CNS2 |
| RC3 | CC4 | LE1 | CNS3 |
| RC4 | CC5 | LE2 | CNS4 |
| RC7 | CC6 | LE3 | CNS5 |
| ME1 | CC7 | LE4 | CNS6 |
| ME2 | BC2 | LE5 | |
| ME4 | BC3 | RC2 | |
| ME7 | BC4 | RC5 | |
| ME8 | BC5 | RC6 | |

To identify the up-regulated pathways in TP53 mutant and wildtype, GSEA was performed using the NCI-60 gene expression data that compares mutant (44 cell lines) versus wild-type (16 cell lines) per KEGG pathway definition. KEGG pathways were rank-sorted by NES of the GSEA. The top pathway identified as up-regulated in the mutant and wildtype cell lines was cell cycle and p53-signaling pathway, respectively, as shown in TABLE 5.

TABLE 5
Top 10 pathways identified by GSEA in TP53 “mutant” versus
“wild-type” of the NCI-60 cell lines.

| | |
|---|---|
| KEGG ID | KEGG Pathway |

Top 10 pathways up-regulated in TP53 mutants

| | |
|---|---|
| HSA04110 | Cell cycle |
| HSA00100 | Biosynthesis of steroids |
| HSA03022 | Basal transcription factors |
| HSA03010 | Ribosome |
| HSA04370 | VEGF signaling pathway |
| HSA04640 | Hematopoietic cell lineage |
| HSA00500 | Starch and sucrose metabolism |
| HSA04660 | T cell receptor signaling pathway |
| HSA03030 | DNA polymerase |
| HSA00770 | Pantothenate and CoA biosynthesis |

Top 10 pathways up-regulated in TP53 wild-type

| | |
|---|---|
| HSA04115 | p53 signaling pathway |
| HSA00120 | Bile acid biosyntehsis |
| HSA00140 | C21-Steroid hormone metabolism |
| HSA00532 | Chondroitin sulfate biosynthesis |
| HSA05030 | Amyotrophic lateral sclerosis (ALS) |
| HSA00531 | Glycosaminoglycan degradation |
| HSA04540 | Gap junction |
| HSA05060 | Prion disease |
| HSA00363 | Bisphenol A degradation |
| HSA04630 | Jak-STAT signaling pathway |

In a recent GSEA study using five different sources of gene-set/pathway definitions (including BioCarta), p53 pathway gene sets were also found to be up-regulated in TP53 wild-types of the NCI-60 panel, in agreement with these results. See Edelman E J, Guinney J, Chi J-T, Febbo P G, Mukherjee S. Modeling cancer progression via pathway dependencies. PLoS Computational Biology 2008; 4(2):e28. The present invention provides methods that are general and flexible, and can be extended to incorporate other gene-set/pathway databases, analysis tools, and new drug screening panels.

### Example 4

### Gene-Expression Versus Pathway-Expression Connections

GS-CMAP according to the present invention was used to connect thirty xenograft pancreatic cancer tumors to NCI-60 panel based both on gene-expression and pathway-expression approaches. Pancreatic cancer is an increasingly prevalent disease with death rates closely mirroring incidence rates, reflecting the ineffectiveness of current therapies. See Jimeno A, Hidalgo M. Molecular biomarkers: their increasing role in the diagnosis, characterization, and therapy guidance in pancreatic cancer. Mol Cancer Ther 5:787-96 (2006). Notably, pancreatic cancer is not included in the NCI-60 panel (see TABLE 3). Using a raw gene expression-based approach the thirty pancreatic tumors connected to four cancer cell lines, two colorectal cancer (CC1: HT29 and CC2: HCC-2998) and two non-small lung cancer cell lines (NSCLC1: NCI-H23 and NSCLC6: NCI-H322M). However, the connections for these pancreatic tumors were more diverse when pathway-expression based approach is used, as shown in TABLE 6.

TABLE 6
Connectivity between 30 xenografts and the top NCI60 cell line.

| | |
|---|---|
| Xenograft Case | Top NCI-60 Cell Line |

(A) Connections based on gene-expression

| | |
|---|---|
| PANC140 | CC2 |
| PANC159 | CC1 |
| PANC163 | CC1 |
| PANC185 | CC1 |
| PANC194 | CC1 |
| PANC198 | NSCLC6 |
| PANC215 | CC2 |
| PANC219 | CC1 |
| PANC247 | CC1 |
| PANC253 | CC2 |
| PANC265 | NSCLC1 |
| PANC266 | CC1 |
| PANC281 | CC1 |
| PANC286 | CC2 |
| PANC287 | CC1 |
| PANC291 | CC1 |
| PANC294 | CC1 |
| PANC354 | CC1 |
| PANC374 | CC2 |
| PANC410 | CC1 |
| PANC420 | CC2 |
| PANC421 | CC1 |
| A6L | CC1 |
| JH010 | CC1 |
| JH011 | CC1 |
| JH015 | CC1 |
| JH024 | CC1 |
| JH027 | CC1 |
| JH033 | CC1 |
| JH034 | CC1 |

(B) Connections based on pathway-expression

| | |
|---|---|
| PANC140 | NSCLC4 |
| PANC159 | CC1 |
| PANC163 | CC2 |
| PANC185 | NSCLC9 |
| PANC194 | RC8 |
| PANC198 | BC4 |
| PANC215 | NSCLC4 |
| PANC219 | ME2 |
| PANC247 | RC8 |
| PANC253 | CC2 |
| PANC265 | BC4 |
| PANC266 | BC4 |
| PANC281 | BC8 |
| PANC286 | BC8 |
| PANC287 | NSCLC9 |
| PANC291 | CC6 |
| PANC294 | NSCLC4 |
| PANC354 | RC5 |
| PANC374 | NSCLC4 |
| PANC410 | RC3 |
| PANC420 | CC2 |
| PANC421 | CC1 |
| A6L | CC1 |
| JH010 | BC8 |
| JH011 | CC1 |
| JH015 | BC8 |
| JH024 | CC1 |
| JH027 | OC6 |
| JH033 | OC6 |
| JH034 | RC5 |

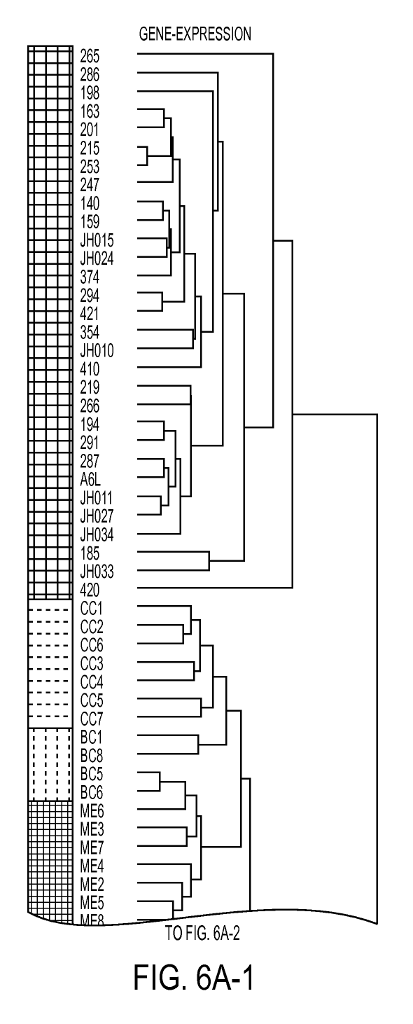

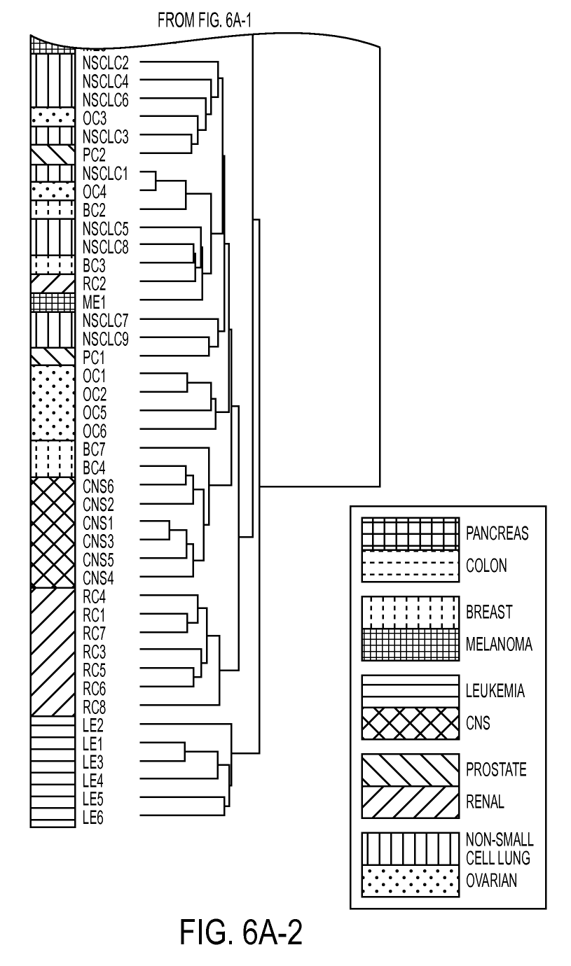

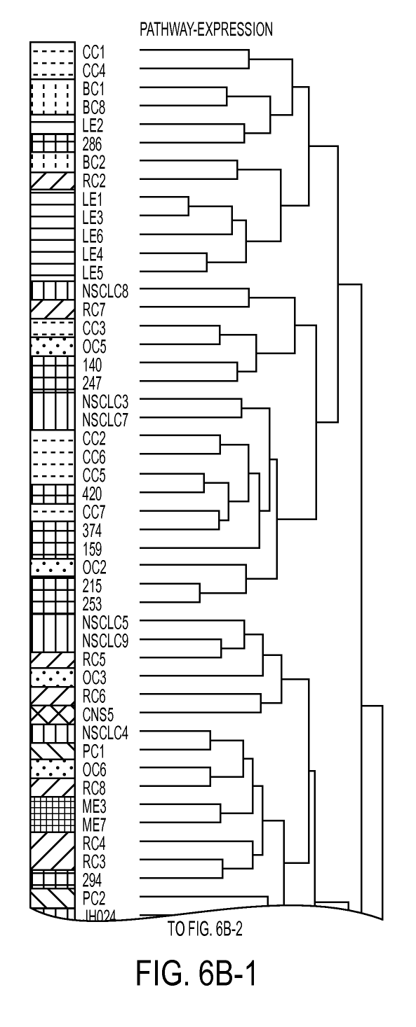

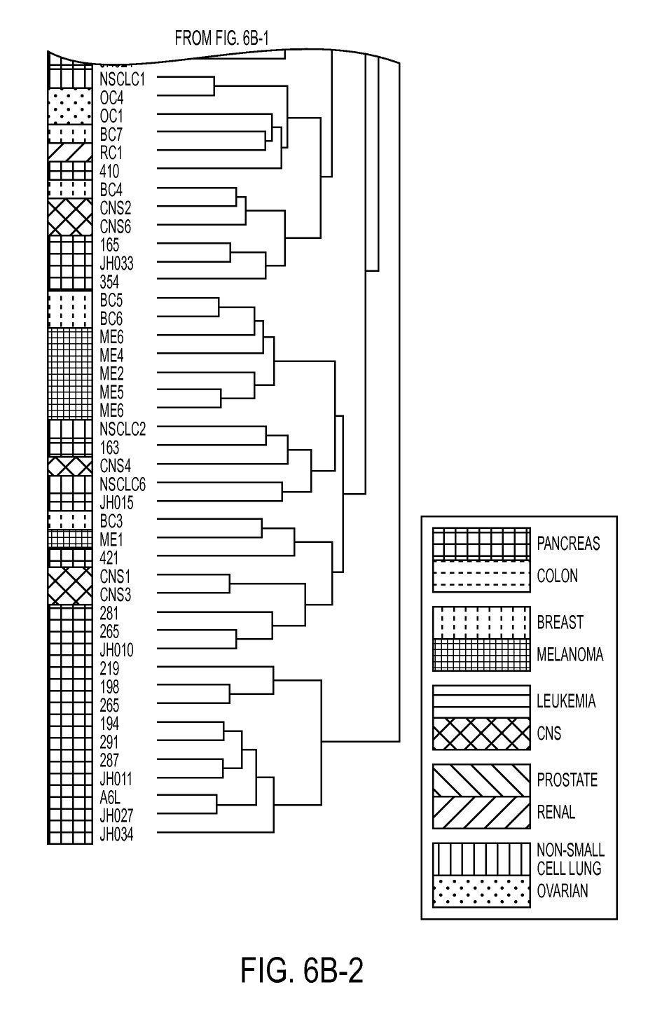

This shows how a molecular mimicry approach can be implemented through this framework. Furthermore, when the pancreatic tumors and NCI-60 panel based on raw gene-expression were clustered, the clusters formed were based on anatomical sites (FIG. 6A). However, the pathway-expression based cluster grouped cancers with similar deregulated pathway patterns regardless on their site of origins (FIG. 6B). In the NCI-60 there are no pancreatic cancer cell lines, showing that drug sensitivity predicted by the present invention depends on molecular profiles rather than on anatomic origin. Pathway-expression provides a non-anatomically but functionally-driven classification of cancer.

### Example 5

### Connecting Gemcitabine Efficacy in GS-CMAP with Xenografts

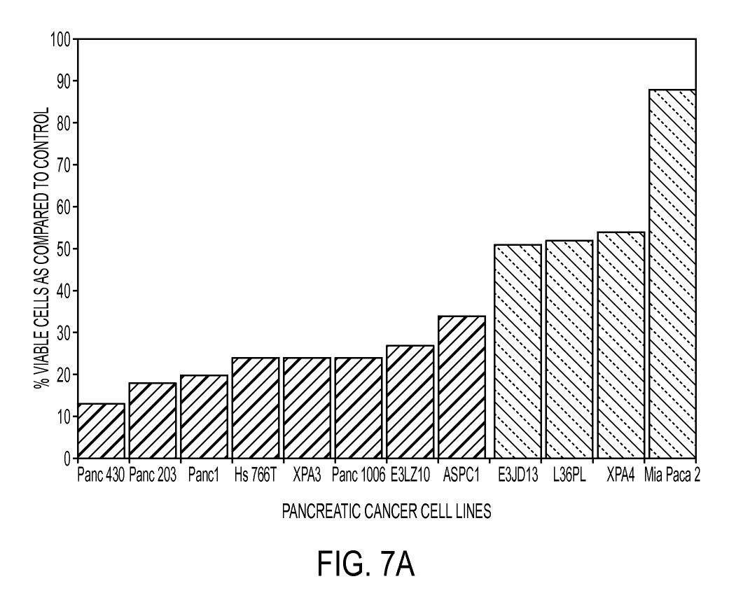

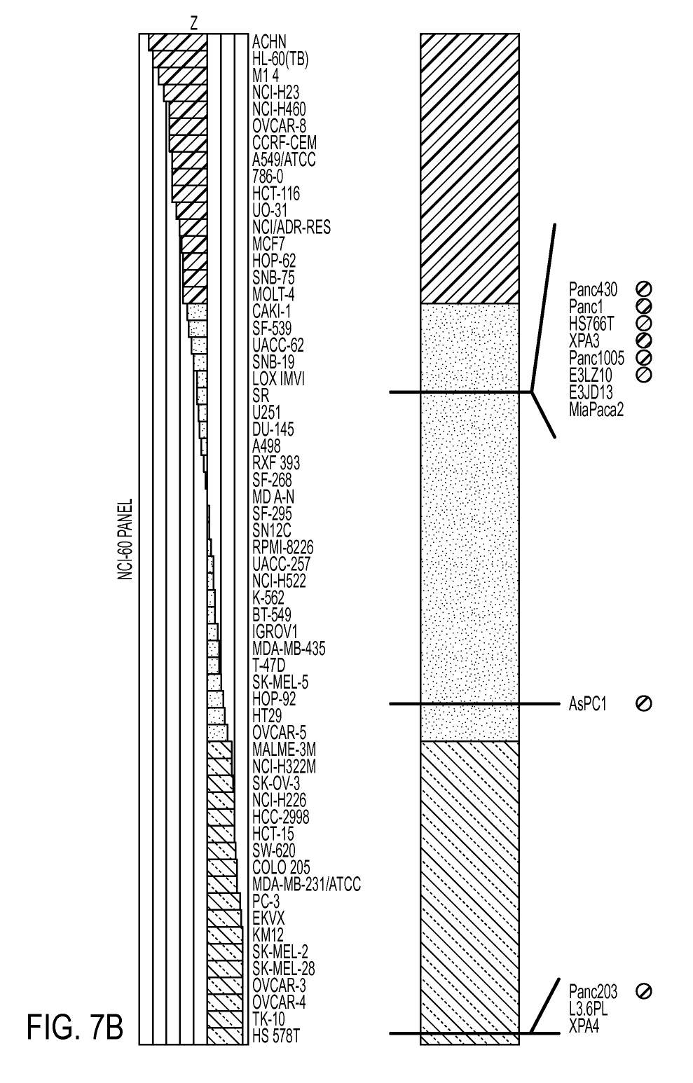

Drug efficacy was determined using connectivity between NCI-60 panel and xenografts. Gemcitabine, a cytotoxic agent, is an approved first-line treatment for pancreatic cancer patients; however, only ˜20% of patients benefit from this therapy. See Herrmann R, Bodoky G, Ruhstaller T, et al. Gemcitabine Plus Capecitabine Compared With Gemcitabine Alone in Advanced Pancreatic Cancer: A Randomized, Multicenter, Phase III Trial of the Swiss Group for Clinical Cancer Research and the Central European Cooperative Oncology Group. Journal of Clinical Oncology 25:2212-17 (2007); Storniolo A M, Enas N H, Brown C A, Voi M, Rothenberg M L, Schilsky R. An investigational new drug treatment program for patients with gemcitabine. Cancer 85:1261-8 (1999). Relative tumor growth inhibition (TGI) values for these tumors were measured after gemcitabine treatment, that ranged from −63% to 94%. When connecting the baseline pathway-expression signatures of the thirty xenografts to GS-CMAP, the sensitive cell lines correlated to five xenografts with an average −24% TGI (FIG. 7). The most sensitive xenograft (PANC194, TGI=−63%) correlated with the top sensitive cell line (RC8: TK-10). A χ²-test was used to determine the statistical significance of the GS-CMAP connections. According to the Response Evaluation Criteria in Solid Tumors (RECIST) criterion, for gemcitabine treatment, tumors with TGI less than −30% were considered as responders in the clinical setting. Following this criterion, the five responders (PANC194, PANC266, PANC294, PANC140 and PANC253) from the thirty xenografts were all correctly connected to sensitive cell lines (χ²-test p-value=0.0027). There is a demonstrable relationship between the TGI value of the xenografts and drug sensitivity of the reference cell lines.

### Example 6

### Predicting and Validating Docetaxel Efficacy in Xenografts with GS-CMAP

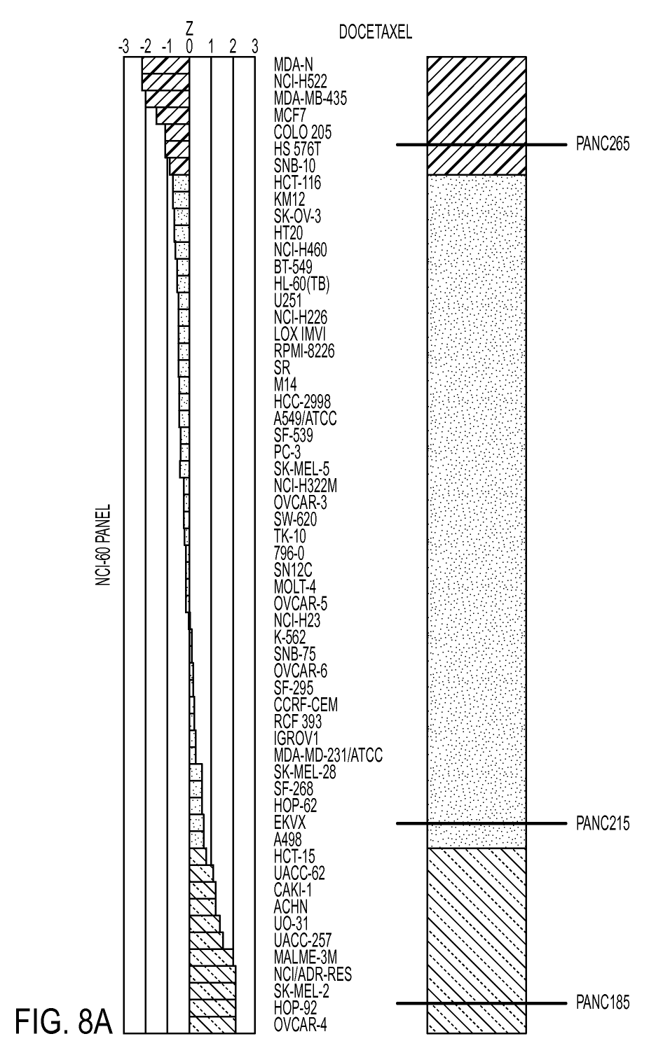

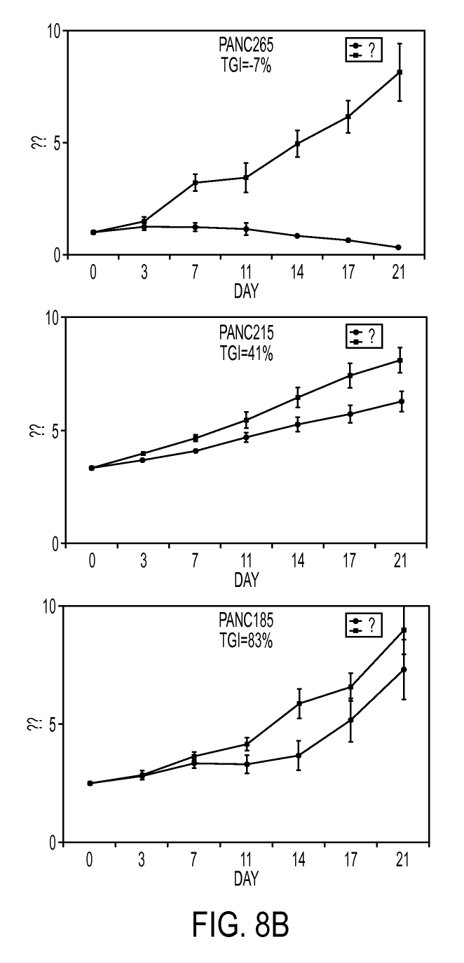

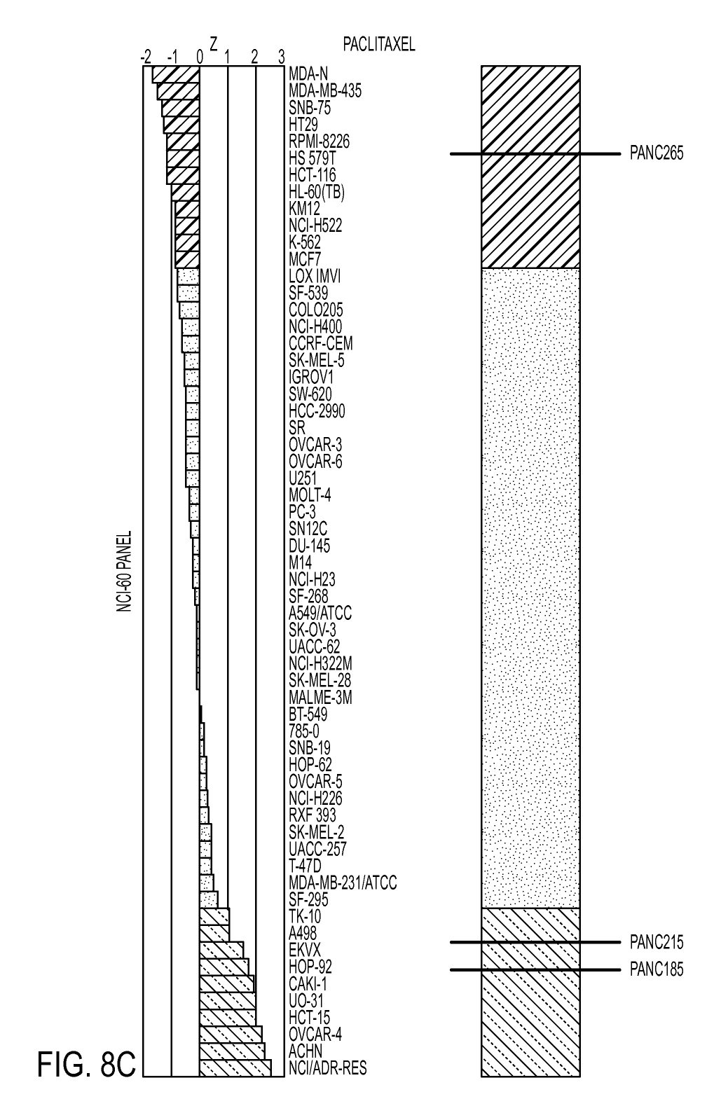

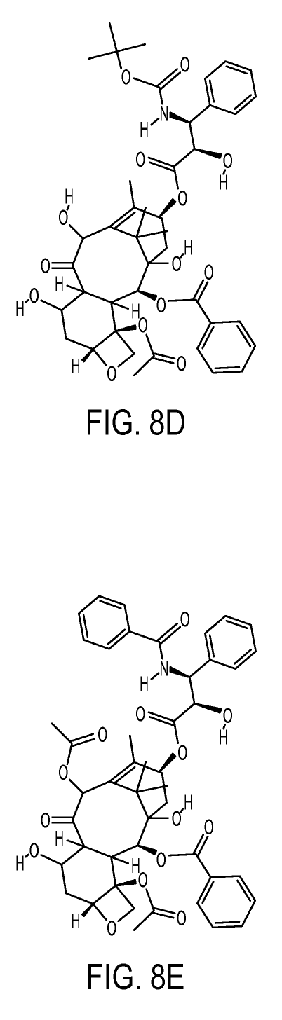

Xenografts connected to the sensitive cell lines in the NCI-60 panel for a particular drug via GS-CMAP are also sensitive to that drug in vivo. To demonstrate this, GS-CMAP was used with direct-patient xenografts in the PancXenoBank. Three xenografts (PANC265, PANC215 and PANC185) were selected that connected to cell lines (HS 578T, EKVX and HOP-92, respectively) with a wide range of sensitivity to docetaxel, and tested these connections in vivo by treating these xenografts with docetaxel for 21 days (FIG. 8). The sensitivity of these xenografts was correctly predicted by the GS-CMAP, as the TGI values for PANC265, PANC215 and PANC185 were −79%, 41% and 83%, respectively (FIG. 4). This shows that the traits determining sensitivity to drugs are not disease specific but biologically-driven, and the present invention can be used to predict treatments for cancers not included in the reference panel.

### Example 7

### Extrapolating Docetaxel Efficacy to Paclitaxel with GS-CMAP

Drug prediction with GS-CMAP can be extrapolated to another structurally related compound. In this example, three xenografts connected previously with docetaxel were treated with paclitaxel. Connecting the xenografts to the paclitaxel sensitivity profile, the same connections were observed as docetaxel, linking PANC265, PANC215 and PANC185 as sensitive, intermediate and resistant to paclitaxel, respectively, as shown in FIG. 8. Thus it is possible to extrapolate the efficacy data from a compound to another structurally related compound.

### Example 8

### Predicting and Validating Rapamycin Efficacy in Xenografts with GS-CMAP

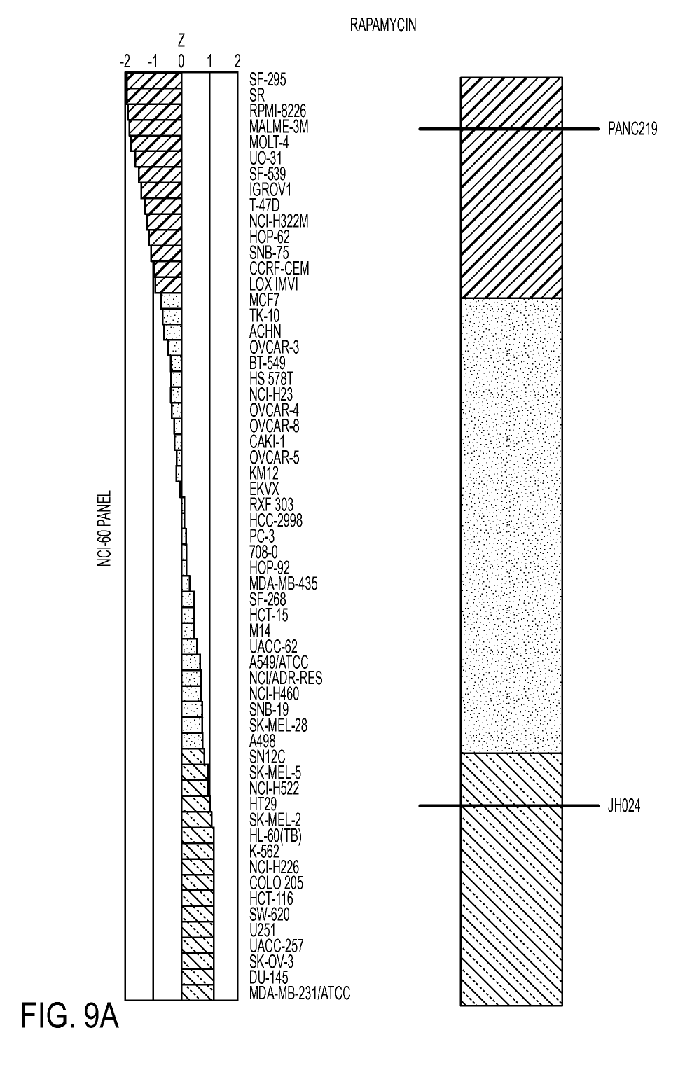

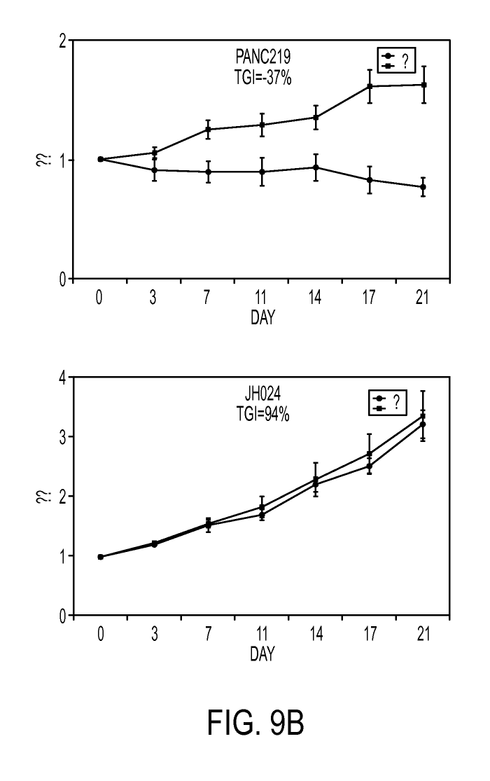

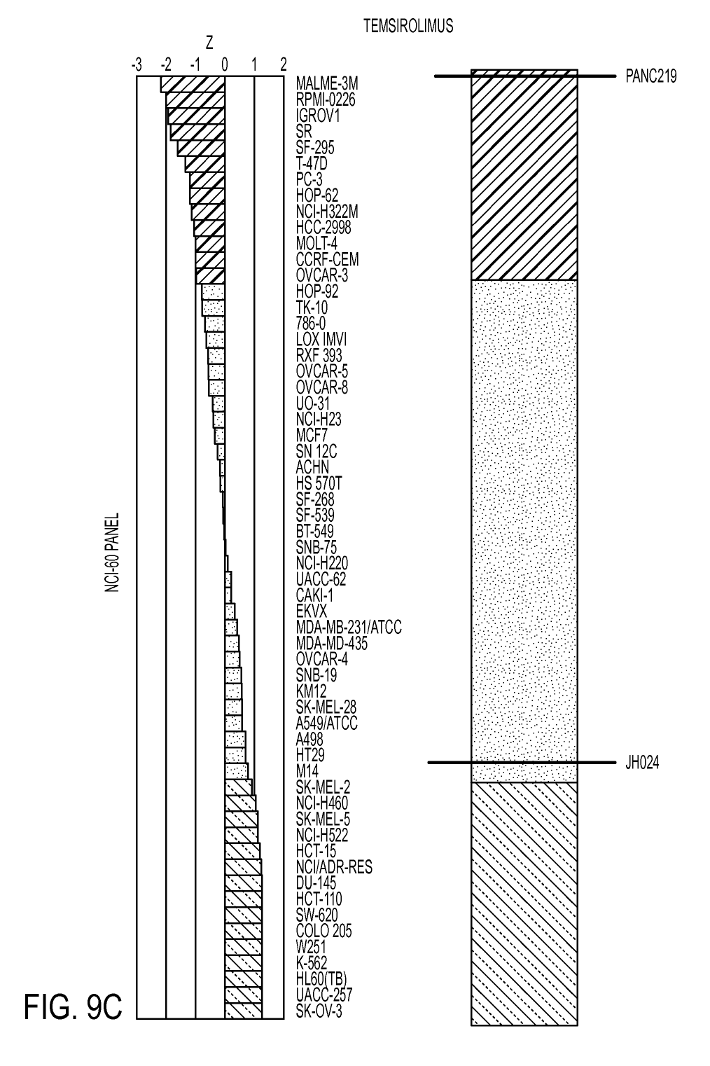

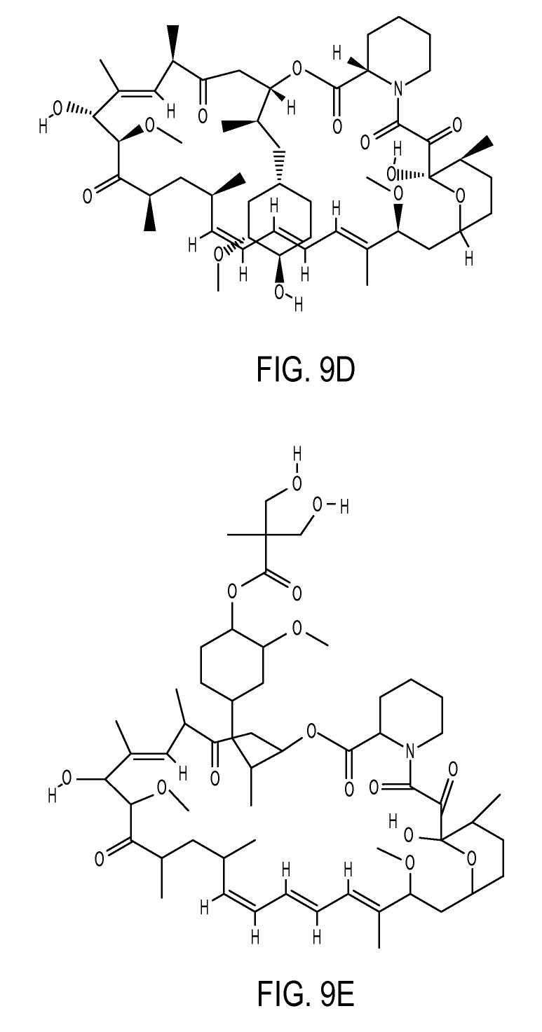

To show that drug efficacy extrapolation is also applicable to targeted agents in addition to cytotoxic drugs, the efficacy of rapamycin on xenografts was queried using the methods of the present invention. The GS-CMAP approach predicted PANC219 as sensitive and JH024 as resistant to rapamycin, respectively (FIG. 9). These xenografts were then treated with temsirolimus, an ester analog of rapamycin with improved pharmaceutical properties and aqueous solubility (and whose sensitivity data is not available in the NCI-60 drug screening panel). The TGI values after temsirolimus treatment were −23% and 94% for PANC219 and JH024, respectively (FIG. 9). Similarly, when these xenograft profiles were connected to temsirolimus, they showed the same drug sensitivities (FIG. 9). This shows that the efficacy extrapolation of one compound to similar compounds or precursor drugs also holds for targeted drugs.

### Example 9

### Connecting Clinical Data with GS-CMAP

The connectivity concept of the present invention can be used with actual patient profiles. Patients were recruited under the Phase II Study of an Individualized Drug Treatment Selection Process Based on a Tumor Xenograft Model for Patients with Resectable Pancreatic Adenocarcinoma (JHOC-J0507 JHOC-05041402, NCT00276744). All patients signed the informed consent of this trial. Twenty-four of the patients with complete follow-up data were included in this study. Direct-patient xenograft cases from the PancXenoBank, Rubio-Viqueira B, Jimeno A, Cusatis G, et al: An In vivo Platform for Translational Drug Development in Pancreatic Cancer. Clin Cancer Res 12:4652-4661, 2006, were also used.

Clinical and pathological characteristics of the patients are shown in TABLE 7.

TABLE 7
Patient Characteristics

| | |
|---|---|
| Characteristics | Sample (%) |

| | | |
|---|---|---|
| Total Patients | 24 | |
| Age, years | | |
| Mean | 65 | |
| Standard Deviation | 10 | |
| Gender | | |
| Male | 11 | (46) |
| Female | 13 | (54) |
| TNM status | | |
| T1N1M0 | 1 | (4) |
| T2N0M0 | 3 | (13) |
| T2N1M0 | 2 | (8) |
| T3N0M0 | 3 | (13) |
| T3N1M0 | 15 | (63) |
| Stage | | |
| I | 4 | (17) |
| II | 5 | (21) |
| III | 15 | (63) |
| Disease free survival, days | | |
| Median | 291 | |
| Range | 44-937 | |

The mean age of diagnosis for these patients is 65 years (range from 41 to 81 years). All patients received gemcitabine as adjuvant chemotherapy. Herrmann R, Bodoky G, Ruhstaller T, et al: Gemcitabine Plus Capecitabine Compared With Gemcitabine Alone in Advanced Pancreatic Cancer: A Randomized, Multicenter, Phase III Trial of the Swiss Group for Clinical Cancer Research and the Central European Cooperative Oncology Group. Journal of Clinical Oncology 25:2212-2217, 2007; Storniolo A M, Enas N H, Brown C A, et al: An investigational new drug treatment program for patients with gemcitabine. Cancer 85:1261-1268, 1999. A patient was defined as resistant to gemcitabine if the disease free survival (DFS) after surgery was less than 300 days. See Oettle H, Post S, Neuhaus P, et al: Adjuvant Chemotherapy with Gemcitabine vs. Observation in Patients Undergoing Curative-Intent Resection of Pancreatic Cancer: A Randomized Controlled Trial. JAMA 297:267-277, 2007. The median DFS for these patients was 291 days.

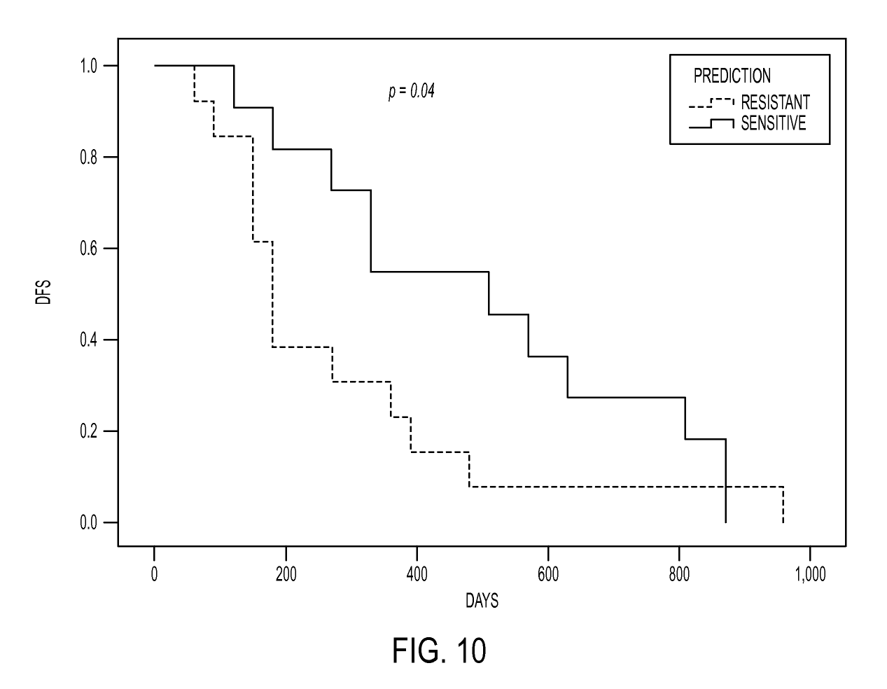

To predict the patient response to gemcitabine, baseline pathway-expression signatures of these patients were connected to GS-CMAP for the gemcitabine sensitivity of NCI-60. Using the methods of the present invention, where a patient is connected to a sensitive cell line, the patient will be predicted as sensitive to gemcitabine and vice versa. This approach achieved an overall prediction accuracy of 71% on these retrospective patient samples. The approach also correctly classified 67% ( 8/12) of the patients that received gemcitabine as adjuvant chemotherapy and showed a stable DFS >300 days (sensitive group) and 75% ( 9/12) of the patients that received gemcitabine as adjuvant chemotherapy and their disease progressed (DFS 300 days, resistant group). The positive predicted value and negative predicted value are 73% and 69%, respectively. The median DFS for the sensitive and resistant groups was 491 and 162 days, respectively. This difference was statistically significant (p=0.04) (FIG. 10).

### Example 10

### Guided Treatment for Gemcitabine Refractory Patients

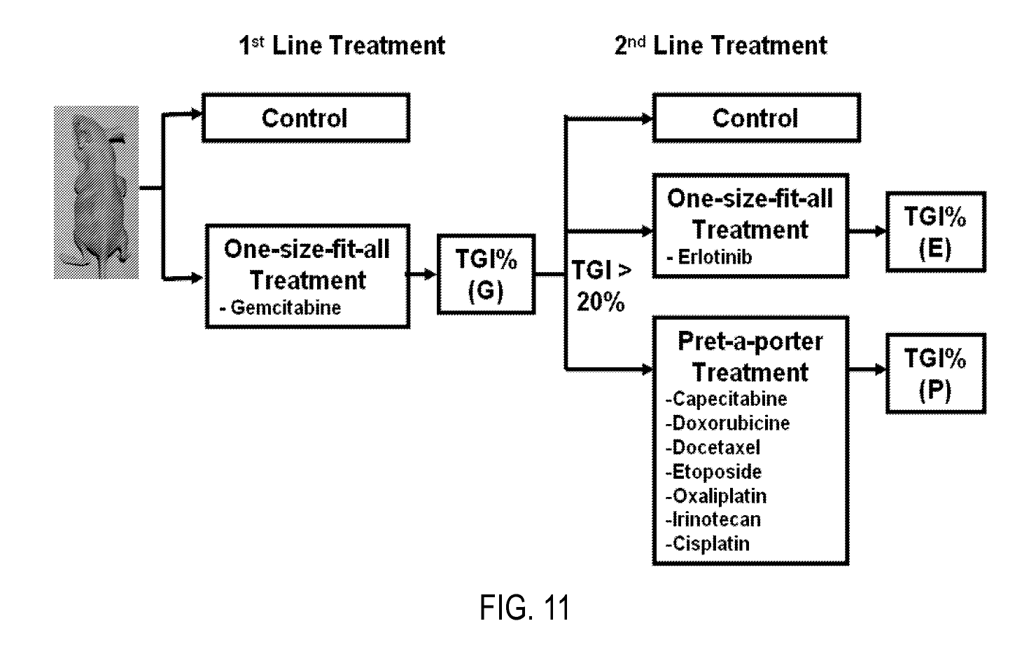

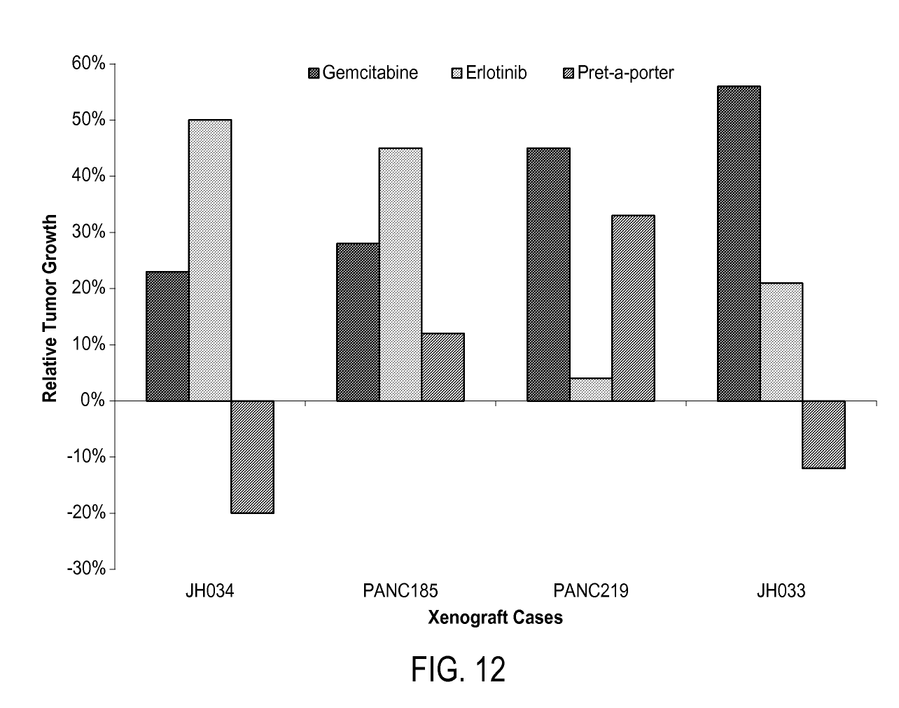

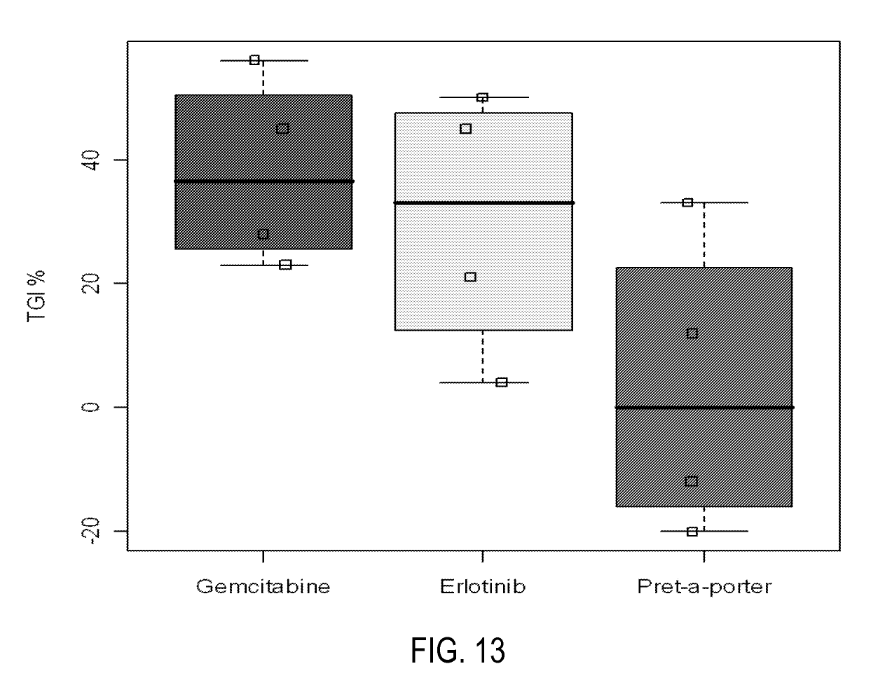

To demonstrate the translational value of this approach, a pre-clinical study was performed comparing patient-derived xenografts refractory to gemcitabine in a second-line treatment of choice of drugs according to the present invention versus the current one-size-fits-all choice of drug (FIG. 11). Seven FDA approved anticancer agents (capecitabine, cisplatin, docetaxel, doxorubicin, etoposide, irinotecan and oxaliplatin) were used as potential second line of drugs for selection according to the present invention. The one-size-fits-all treatment was erlotinib, an FDA approved targeted therapy for advanced pancreatic cancer. See Moore M J, Goldstein D, Hamm J, et al. Erlotinib plus gemcitabine compared with gemcitabine alone in patients with advanced pancreatic cancer: a phase Ill trial of the National Cancer Institute of Canada Clinical Trials Group. J Clin Oncol 25:1960-06 2007. Four xenograft cases (PANC185, PANC219, JH033 and JH034) that showed more than 20% TGI when treated with gemcitabine were selected in this study. Xenograft models of these cases were randomly assigned to (i) control, (ii) erlotinib arm (one-size-fits-all second line treatment) and (iii) molecular mimicry arm (i.e., drug selection according to the present invention). For the molecular mimicry arm, the baseline gene expression profiles of these cases were connected the NCI reference panel using GS-CMAP, and one of the seven anticancer agents was assigned to the case based on comparing the GI₅₀ among these drugs. Molecular mimicry predicted that JH033 and JH034 are sensitive to docetaxel, PANC185 is sensitive to irinotecan and PANC219 is sensitive to doxorubicin. These cases were treated with the molecular mimicry choice of drugs and tumor volumes were measured. At the end of the experiment, xenografts treated with molecular mimicry choice of drugs had higher tumor growth inhibition than their litter mates treated with erlotinib (FIGS. 12 and 13). Regression in tumor volume was observed in two cases (JH033 and JH034) when treated with the molecular mimicry choice of drugs (FIG. 12). The mean TGI % for the molecular mimicry tumors was about 10-fold lower than the erlotinib arm (3% versus 30%, Chi-square test, p=0.02) (FIG. 13).

### Example 11

### Proteomics Approach

A reference database is created by determining a proteomics profile for each of the NCI-60 cell lines using high-density reverse-phase lysate microarrays. See Nishizuka et al., Proteomic profiling of the NCI-60 cancer cell lines using new high-density reverse-phase lysate microarrays. Proc Nat'l Acad Sci USA 100: 14229-14234 (2003). The database links the proteomics profile from each NCI-60 cell line to the corresponding drug sensitivity data previously available in the same manner that gene expression profile is linked. The proteomics profile of a biological sample is determined using a high-density reverse-phase lysate microarray. The sample is connected to the reference panel by correlating the sample's proteomics profile versus those of the reference panel.

### Example 12

### Perturbability Assay

Direct pancreatic cancer derived xenografts: Resected pancreatic adenocarcinomas are routinely implanted in nude mice at the Johns Hopkins Medical Institutions as a method to obtain enriched populations of neoplastic cells under an IRB-approved protocol from residual pancreatic cancer tumors. Tumor specimens from Whipple resection specimens were divided into 2-3 mm³ pieces in antibiotic-containing RPMI media. Pieces of non-necrotic tissue were selected and immersed in Matrigel. Under anesthesia with isofluorane, tumors were implanted into five-to-six week-old female athymic (nu/nu) mice purchased from Harlan (Harlan Laboratories, Washington, D.C.). This research protocol was approved by the Johns Hopkins University Animal Use and Care Committee arid animals were maintained in accordance to guidelines of the American Association of Laboratory Animal Care. Tumors were propagated to subsequent cohorts of mice until a sufficient number were available for drug testing.

Fine needle aspiration (FNA): FNAs on mice were performed according to standard cytopathologic practice using 10 cc syringes and 25-gauge needles. The procedure was performed under inhaled general anesthesia with isofluorane. During each FNA procedure the first pass was smeared onto glass slides and used for morphologic analysis and quality control, (DiffQuik™ and Papanicoloau), and the second through sixth passes were used to acquire viable cells.

Ex vivo assay: FNA passes used to acquire viable cells were immediately transferred into 10 ml sterile prewarmed complete RPMI-1640 culture medium containing 10% FBS, penicillin (200 ug/ml), and streptomycin (200 ug/ml). Equivalent aliquots of cells were seeded in two wells of a 6-well polypropylene microplate. Cells were treated with vehicle or gemcitabine in a humidified 5% CO₂ incubator at 37° C. for 1-24 hours, at a concentration of 1-10 μM. Following treatment, non-adherent and adherent cells (collected by scraping) were pooled together in a 1.5 mL microcentrifuge tube and centrifuged at 500 g for 5 mm at 4° C. After washing with PBS, cells were lysed in 100 μL of ice-cold RLT.

RNA extraction and cDNA generation: Total RNA was extracted from cells using the RNeasy™ Mini Kit (Qiagen, Valencia, Calif.) following the manufacturer's instructions. cDNA was synthesized using iScript cDNA synthesis kit (Bio-Rad) following the manufacturer's instructions.

Low-density micro arrays (LDMA): The samples were run in a customized assay with a 384-well format (ABI, Foster City, Calif.), where the expression of 45 to 381 relevant genes can be quantitatively tested. For this example, the array was designed with 8 replicated sections containing 48 wells, with primers of one gene per well: 3 housekeeping genes (HK: UBC, HPRT, SDHA), and 45 genes of interest. 50 μL of cDNA plus 50 μL of ABI mastermix were loaded onto each of two contiguous lanes to have duplicated readouts of every sample, and were run by real-time reverse transcriptase PCR (RT-PCR). The amount of change in the target genes was calculated by comparing the threshold cycle (CT) of each gene to the geometric mean of the CTs of the HK genes. Relative expression of the target genes was estimated using the formula: relative expression=2−^{ΔCT}, where ΔCT=CT (target gene)−CT (average of the 3 HK genes). The experiment was run in duplicate and mean values are analyzed.

Data analysis: The expression level of each gene in the treated sample was normalized to that of the untreated control. A significant change was defined when the gene expression either increases two-fold or more (>200%), or decreases to half or more (<50%). The rest is considered uninformative.

Results: This assay was used to profile a series of direct pancreatic cancer xenografts, using a LDMA customized with relevant genes in pancreatic cancer. Fine needle aspiration biopsies on 10 tumors corresponding to 10 different cases were used, each from a different patient. All 10 procedures rendered sufficient cell material to adequately conduct the ex vivo exposure, and yielded sufficient RNA to run the assay within the manufacturer's specifications in terms of cDNA amount.

TABLE 8 depicts the data obtained from this experiment. A target against which a drug was available was identified in 9 of 10 assayed cases. Half of the genes did not show any significant variation in any of the ten cases, and of the 400+ data items less than 10% showed a significant change. These observations indicate that the drug-induced perturbation caused specific and relevant changes in gene expression, and not unspecific variations.

Here, a series of targets (PLK1, AXIN2, CXCR4, IGFBP3) were identified as upregulated in pancreatic cancer. Some of them were known to be, but others were not.

TABLE 8
Gene expression upon gemcitabine-induced ex vivo perturbation.

| | #1 | #2 | #3 | #4 | #5 | #6 | #7 | #8 | #9 | #10 |
|---|---|---|---|---|---|---|---|---|---|---|
| CyclinB1 | 68 | 99 | 48 | 124 | 53 | 84 | 16 | 140 | 115 | 89 |
| TGFA | 99 | 113 | 92 | 85 | 97 | 101 | 131 | 107 | 103 | 141 |
| HIF1A | 126 | 111 | 106 | 146 | 108 | 102 | 115 | 113 | 100 | 90 |
| Survivin | 81 | 110 | 58 | 166 | 67 | 145 | 85 | 74 | 104 | 64 |
| AXIN2 | 67 | 63 | 44 | 142 | 210 | 517 | 41 | 81 | 106 | 202 |
| MSLN | 101 | 118 | 91 | 89 | 100 | 101 | 117 | 98 | 107 | 90 |
| FOS | 113 | 115 | 123 | 108 | 112 | 106 | 139 | 113 | 83 | 96 |
| BRCA1 | 81 | 63 | 84 | 343 | 95 | 195 | 89 | 59 | 161 | 62 |
| RRM1 | 110 | 166 | 81 | 79 | 92 | 114 | 129 | 104 | 73 | 92 |
| BRAF | 116 | 98 | 93 | 175 | 146 | 88 | 40 | 122 | 93 | 65 |
| GADD45A | 108 | 108 | 104 | 78 | 121 | 75 | 96 | 147 | 74 | 106 |
| SHH | 70 | 108 | 65 | 91 | 113 | 116 | 57 | 77 | 47 | 70 |
| NFKB1 | 114 | 138 | 94 | 104 | 163 | 83 | 105 | 105 | 110 | 78 |
| p21 | 96 | 110 | 99 | 102 | 143 | 122 | 134 | 100 | 83 | 119 |
| BNIP3L | 109 | 96 | 82 | 115 | 90 | 105 | 100 | 106 | 95 | 97 |
| NOTCH2 | 113 | 119 | 103 | 92 | 56 | 79 | 112 | 122 | 101 | 101 |
| VHL | 104 | 116 | 118 | 127 | 79 | 111 | 80 | 101 | 110 | 74 |
| BAX | 91 | 95 | 78 | 100 | 109 | 110 | 100 | 91 | 92 | 91 |
| DCK | 164 | 112 | 80 | 176 | 132 | 91 | 102 | 99 | 117 | 91 |
| HIF1B | 99 | 129 | 106 | 56 | 65 | 65 | 102 | 566 | 107 | 76 |
| EGFR | 124 | 101 | 70 | 54 | 71 | 74 | 151 | 91 | 85 | 50 |
| CCND1 | 127 | 128 | 106 | 171 | 141 | 85 | 127 | 107 | 97 | 69 |
| GLI2 | 72 | 82 | 73 | 11 | 118 | 85 | 73 | 531 | 99 | 30 |
| MAPK3 | 152 | 71 | 109 | 108 | 95 | 75 | 167 | 90 | 106 | 73 |
| PLK1 | 50 | 34 | 36 | 15 | 21 | 31 | 98 | 90 | 111 | 68 |
| NOTCH1 | 158 | 138 | 132 | 134 | 89 | 60 | 104 | 118 | 96 | 70 |
| IGF1R | 51 | 105 | 66 | 41 | 61 | 82 | 28 | 101 | 53 | 66 |
| PSCA | 106 | 127 | 115 | 81 | 102 | 132 | 91 | 101 | 94 | 108 |
| VEGF | 106 | 101 | 86 | 103 | 107 | 91 | 115 | 105 | 87 | 97 |
| PTCH | 81 | 102 | 53 | 93 | 48 | 153 | 63 | 203 | 78 | 33 |
| CXCR4 | 100 | 5 | 43 | 13 | 229 | 0 | 5 | 342 | 649 | 53 |
| ERCC1 | 107 | 78 | 88 | 94 | 105 | 150 | 153 | 98 | 91 | 91 |
| IGFBP3 | 135 | 205 | 49 | 130 | 122 | 34 | 78 | 83 | 42 | 96 |
| MAPK1 | 114 | 104 | 111 | 68 | 86 | 90 | 93 | 78 | 99 | 79 |
| MAP2K2 | 127 | 78 | 106 | 146 | 151 | 98 | 131 | 86 | 90 | 89 |
| JUN | 118 | 109 | 114 | 83 | 135 | 106 | 80 | 108 | 92 | 82 |
| MDM2 | 134 | 92 | 137 | 43 | 73 | 81 | 180 | 65 | 92 | 86 |
| IHH | 113 | 131 | 130 | 92 | 141 | 122 | 81 | 137 | 99 | 93 |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for selecting a candidate therapeutic agent, comprising:
(a) determining a gene set expression profile for two or more genes in a target cell;
(b) comparing the gene set expression profile of the target cell to one or more gene set expression profiles of a panel of reference cells, wherein the panel comprises cells from more than two different cell types;
(c) identifying a reference cell from the panel that has the most similar gene set expression profile to the target cell according to the comparison in step b); and
(d) selecting a therapeutic agent known for treating a condition in the reference cell identified in step c).

2. The method of claim 1 wherein determining the gene set expression profile of the target cell comprises amplifying nucleic acids extracted from the target cell by reacting the nucleic acids with a plurality of nucleotide probes.

3. The method of claim 2 wherein the reaction products are hybridized to one or more DNA microarrays.

4. The method of claim 2 wherein the amplification comprises a real-time polymerase chain reaction.

5. The method of claim 1 wherein the gene set expression profile of the target cell is determined using protein expression levels.

6. The method of claim 1 wherein determining the gene set expression profile of the target cell comprises comparing the expression levels of pre-defined gene sets in the target cell against the expression levels of the same gene sets in the panel of reference cells.

7. The method of claim 1 wherein the gene sets comprise biological pathways.

8. The method of claim 1 wherein the target cell is extracted from a mammalian subject.

9. The method of claim 8 wherein the extraction is from a tumor biopsy.

10. The method of claim 9 wherein the biopsy comprises a fine needle aspirate biopsy, a paraffin block, or a frozen sample.

11. The method of claim 1 wherein the panel of reference cells comprises one or more cells listed in Table 3.

12. A method for treating a subject in need thereof, comprising:
(a) extracting a sample from the subject;
(b) determining a gene set expression profile for two or more genes in a target cell derived from the sample in step a);
(c) comparing the gene set expression profile of the target cell to one or more gene set expression profiles of a panel of reference cells, wherein the panel comprises cells from more than two different cell types;
(d) identifying a reference cell from the panel that has the most similar gene set expression profile to the target cell according to the comparison in step c); and
(e) treating the subject with one or more therapeutic agents known for treating a condition in the reference cell identified in step d).

13. A method for selecting a candidate therapeutic agent comprising:
(a) contacting a target cell with a first therapeutic agent;
(b) determining a response of the target cell to the first therapeutic agent using expression profiling; and
(c) selecting a second therapeutic agent based on the response of the target cell to the first therapeutic agent.

14. The method of claim 13 wherein the target cell has not previously been contacted with the first therapeutic agent.

15. The method of claim 13 wherein the expression profiling comprises amplifying nucleic acid extracted from the target cell by reacting the nucleic acid with a plurality of nucleotide probes.

16. The method of claim 15 wherein the reaction products are hybridized to one or more DNA microarrays.

17. The method of claim 15 wherein the amplification comprises a real-time polymerase chain reaction.

18. The method of claim 13 wherein determining the response of the target cell to the first therapeutic agent comprises:
i) determining the expression level of multiple genes in the target cell after contacting the target cell with the first therapeutic agent;
ii) determining the expression level of the same genes in an identical control cell that has not been contacted with the first therapeutic agent;
iii) comparing the expression levels determined in step i) and step ii); and
iv) identifying genes that are overexpressed or underexpressed in the target cell versus the control cell according to the comparison in step iii).

19. The method of claim 18 wherein the second therapeutic agent selected in step c) is a known therapeutic for cells that overexpress or underexpress one of more of the genes identified in step iv).

20. The method of claim 18 wherein the set of multiple genes whose expression is determined comprises one or more genes listed in Table 8.
